(19) United States
(12) Patent Application Publication
(10) Pub. No.: US 2026/0226488 A1
(43) Pub. Date: Aug. 6, 2026

(54) METHOD FOR INCREASING SOYBEAN SEED SIZE

(71) Applicant: SHANDONG SHUNFENG BIOTECHNOLOGY CO.LTD., Jinan (CN)

(72) Inventors: Hongtao XIE, Jinan (CN); Zhanbo JI, Jinan (CN); Kang PANG, Jinan (CN)

(73) Assignee: SHANDONG SHUNFENG BIOTECHNOLOGY CO.LTD., Jinan (CN)

(21) Appl. No.: 19/151,348

(22) PCT Filed: Mar. 15, 2024
(86) PCT No.: PCT/CN2024/081818

§ 371 Date: Jul. 28, 2025

(30) Foreign Application Priority Data
Mar. 23, 2023 (CN) 202310304497.5

(51) Int. Cl. C12N 15/82; C12N 9/22; C12N 15/11; C12N 15/113

(52) U.S. Cl. CPC C12N 15/8261; C12N 9/226; C12N 15/11; C12N 15/113; C12N 15/8213; C12N 2310/141; C12N 2310/20

(57) ABSTRACT

A method for increasing a soybean seed size is provided. In the method, microRNA396 (miR396) in soybean is edited with Cas12i and gRNA to produce soybean with an improved seed size and yield. The mutant miR396 undergoes a base mutation relative to parental miR396; the parental miR396 is WT miR396 derived from soybean; the miR396 is selected from any one or more of miR396a, miR396b, miR396c, miR396d, miR396f, and miR396i.

### CROSS REFERENCE TO THE RELATED APPLICATIONS

This application is the national phase entry of International Application No. PCT/CN2024/081818, filed on Mar. 15, 2024, which is based upon and claims priority to Chinese Patent Application No. 202310304497.5, filed on Mar. 23, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBSDSF015-PKG_Sequence_Listing.xml, created on Jul. 17, 2025, and is 26,274 bytes in size.

### TECHNICAL FIELD

The present disclosure belongs to the field of biotechnology, and specifically relates to a method for gene editing in soybean. In particular, the present disclosure relates to a mutant microRNA396 (miR396) produced by a Cas12i gene editing technology and a use of the mutant miR396 for increasing a soybean seed size.

### BACKGROUND

Soybean is a major source for fats and proteins in the human, and is also an important source of vegetable oils and plant proteins for the human. Soybean is one of the most significant cash crops worldwide. Soybeans are widely used to produce bean products and feeds due to their advantages such as comprehensive nutrition and high protein content. However, due to low soybean outputs and poor economic returns of soybean cultivation for farmers in China, the soybean cultivation area remains limited in China, with the total production merely of 20 million tons. As a result, 80% or more of soybeans in China rely on imports, posing a serious threat to national food security.

microRNAs (miRNAs) are a class of non-coding single-stranded small RNA molecules. miRNAs can bind to mRNAs of target genes through base pairing, thereby regulating the target genes. During the growth of plants, plant miRNAs play critical roles in regulating the characteristics of meristematic tissues, the leaf polarity, the flowering patterns, etc. Plant miR396 is a highly-conserved endogenous non-coding single-stranded small RNA molecule with a length of 20 to 24 nucleotides, which can regulate the expression of a growth-regulating factor (GRF) for a target gene. Studies have shown that miR396 can regulate the tillering, grain type, and spike type of a crop to affect the output of the crop.

When used for crop breeding, the clustered regularly interspaced short palindromic repeats (CRISPR)/Cas gene editing technology offers advantages such as no transgenic components, short breeding cycle, and directed modification of plant traits. The CRISPR/Cas Type V system is a newly discovered CRISPR system. The CRISPR/Cas Type V system has a motif of 5′-TTN and can cleave a sticky end of a target sequence, such as Cpf1, C2c1, CasX, and CasY. However, the different CRISPR/Cas systems currently available have distinct advantages and disadvantages. Cas12i is also a CRISPR/Cas Type V system. The Chinese patent (CN111757889B, publication date: May 25, 2021) discloses a Type V Cas enzyme (Cas12f.4). In the present disclosure, Cas12f.4 is defined as Cas12i. The editing efficiency of Cas12i for gene editing in eukaryotic cells still needs to be improved.

In the present disclosure, genes of the soybean miR396 family are directly edited through optimized Cas12i to develop germplasm resources of large-seed and high-yield soybean.

### SUMMARY

An objective of the present disclosure is to provide a mutant miR396 and a use of the mutant miR396 for increasing a soybean seed size, enhancing a soybean seed weight, increasing a number of seeds per soybean plant, or improving a soybean yield.

In an aspect, the present disclosure provides a mutant miR396, where the mutant miR396 undergoes a base mutation relative to parental miR396; the parental miR396 is wild-type (WT) miR396 derived from soybean; and the miR396 is selected from any one or more of miR396a, miR396b, miR396c, miR396d, miR396f, and miR396i.

In an embodiment, the miR396 includes the miR396a, the miR396c, and the miR396d.

In an embodiment, the miR396 includes the miR396a, the miR396c, the miR396d, and the miR396f.

In an embodiment, the miR396 includes the miR396c, the miR396d, and the miR396f.

In an embodiment, the miR396 includes the miR396a, the miR396b, the miR396c, the miR396d, and the miR396f.

In an embodiment, the miR396 includes the miR396a, the miR396c, and the miR396i.

In an embodiment, the miR396 includes the miR396a, the miR396d, and the miR396f.

In an embodiment, the miR396 includes the miR396a, the miR396b, the miR396c, and the miR396d.

In an embodiment, the miR396 includes the miR396a, the miR396c, the miR396f, and the miR396i.

In an embodiment, the miR396 includes the miR396b, the miR396c, the miR396d, the miR396f, and the miR396i.

In an embodiment, the mutant miR396 is selected from any one or more of the following (i) to (ix):
(i) miR396a, miR396c, and miR396d that undergo a base mutation relative to parental miR396;
(ii) miR396a, miR396c, miR396d, and miR396f that undergo a base mutation relative to parental miR396;
(iii) miR396c, miR396d, and miR396f that undergo a base mutation relative to parental miR396;
(iv) miR396a, miR396b, miR396c, miR396d, and miR396f that undergo a base mutation relative to parental miR396;
(v) miR396a, miR396c, and miR396i that undergo a base mutation relative to parental miR396;
(vi) miR396a, miR396d, and miR396f that undergo a base mutation relative to parental miR396;
(vii) miR396a, miR396b, miR396c, and miR396d that undergo a base mutation relative to parental miR396;
(viii) miR396a, miR396c, miR396f, and miR396i that undergo a base mutation relative to parental miR396; and
(ix) miR396b, miR396c, miR396d, miR396f, and miR396i that undergo a base mutation relative to parental miR396.

In an embodiment, the mutant miR396 is able to improve a seed size, a seed weight, a number of seeds per plant, or a yield for soybean.

In an embodiment, the base mutation includes a base deletion, a base insertion, or a base substitution.

In an embodiment, the parental miR396 is the WT miR396 derived from the soybean. In an embodiment, the soybean is Zhonghuang 302.

In an embodiment, parental miR396a is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 2.

In a specific embodiment, a sequence of the parental miR396a is shown in SEQ ID NO: 2.

In an embodiment, parental miR396b is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 3.

In a specific embodiment, a sequence of the parental miR396b is shown in SEQ ID NO: 3.

In an embodiment, parental miR396c is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 4.

In a specific embodiment, a sequence of the parental miR396c is shown in SEQ ID NO: 4.

In an embodiment, parental miR396d is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 5.

In a specific embodiment, a sequence of the parental miR396d is shown in SEQ ID NO: 5.

In an embodiment, parental miR396f is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 6.

In a specific embodiment, a sequence of the parental miR396f is shown in SEQ ID NO: 6.

In an embodiment, parental miR396i is derived from soybean, and has a sequence identity of at least 80%, at least 85%, at least 90%, at least 91%, at least 92%, at least 93%, at least 94%, at least 95%, at least 96%, at least 97%, at least 98%, or at least 99% with a sequence shown in SEQ ID NO: 7.

In a specific embodiment, a sequence of the parental miR396i is shown in SEQ ID NO: 7.

In an embodiment, the mutant miR396a undergoes a base mutation relative to WT miR396a, the mutant miR396b undergoes a base mutation relative to WT miR396b, the mutant miR396c undergoes a base mutation relative to WT miR396c, the mutant miR396d undergoes a base mutation relative to WT miR396d, the mutant miR396f undergoes a base mutation relative to WT miR396f, and the mutant miR396i undergoes a base mutation relative to WT miR396i.

In an embodiment, the mutant miR396a undergoes a base deletion relative to the sequence shown in SEQ ID NO: 2; the mutant miR396b undergoes a base deletion relative to the sequence shown in SEQ ID NO: 3; the mutant miR396c undergoes a base deletion relative to the sequence shown in SEQ ID NO: 4; the mutant miR396d undergoes a base deletion relative to the sequence shown in SEQ ID NO: 5; the mutant miR396f undergoes a base deletion relative to the sequence shown in SEQ ID NO: 6; and the mutant miR396i undergoes a base deletion relative to the sequence shown in SEQ ID NO: 7.

In an embodiment, the sequence of the parental miR396a is shown in SEQ ID NO: 2, and the mutant miR396a undergoes deletions of 229th to 233rd bases relative to the sequence shown in SEQ ID NO: 2; the sequence of the parental miR396c is shown in SEQ ID NO: 4, and the mutant miR396c undergoes deletions of 229th to 230th bases relative to the sequence shown in SEQ ID NO: 4; and the sequence of the parental miR396d is shown in SEQ ID NO: 5, and the mutant miR396d undergoes deletions of 19th to 29th bases relative to the sequence shown in SEQ ID NO: 5.

In an embodiment, the sequence of the parental miR396a is shown in SEQ ID NO: 2, and the mutant miR396a undergoes a deletion of a 229th base relative to the sequence shown in SEQ ID NO: 2; the sequence of the parental miR396c is shown in SEQ ID NO: 4, and the mutant miR396c undergoes deletions of 230th to 255th bases relative to the sequence shown in SEQ ID NO: 4; the sequence of the parental miR396d is shown in SEQ ID NO: 5, and the mutant miR396d undergoes deletions of 19th to 29th bases relative to the sequence shown in SEQ ID NO: 5; and the sequence of the parental miR396f is shown in SEQ ID NO: 6, and the mutant miR396f undergoes a deletion of a 265th base relative to the sequence shown in SEQ ID NO: 6.

In an embodiment, the sequence of the parental miR396c is shown in SEQ ID NO: 4, and the mutant miR396c undergoes deletions of 228th to 253rd bases relative to the sequence shown in SEQ ID NO: 4; the sequence of the parental miR396d is shown in SEQ ID NO: 5, and the mutant miR396d undergoes a deletion of a 19th base relative to the sequence shown in SEQ ID NO: 5; and the sequence of the parental miR396f is shown in SEQ ID NO: 6, and the mutant miR396f undergoes deletions of 264th to 265th bases relative to the sequence shown in SEQ ID NO: 6.

In an embodiment, the sequence of the parental miR396a is shown in SEQ ID NO: 2, and the mutant miR396a undergoes a deletion of a 229th base relative to the sequence shown in SEQ ID NO: 2; the sequence of the parental miR396b is shown in SEQ ID NO: 3, and the mutant miR396b undergoes deletions of 30th to 100th bases relative to the sequence shown in SEQ ID NO: 3; the sequence of the parental miR396c is shown in SEQ ID NO: 4, and the mutant miR396c undergoes a deletion of a 229th base relative to the sequence shown in SEQ ID NO: 4; the sequence of the parental miR396d is shown in SEQ ID NO: 5, and the mutant miR396d undergoes deletions of 18th to 26th bases relative to the sequence shown in SEQ ID NO: 5; and the sequence of the parental miR396f is shown in SEQ ID NO: 6, and the mutant miR396f undergoes deletions of 264th to 265th bases relative to the sequence shown in SEQ ID NO: 6.

In an embodiment, the sequence of the parental miR396a is shown in SEQ ID NO: 2, and the mutant miR396a undergoes deletions of 229th to 233rd bases relative to the sequence shown in SEQ ID NO: 2; the sequence of the parental miR396c is shown in SEQ ID NO: 4, and the mutant miR396c undergoes deletions of 228th to 253rd bases relative to the sequence shown in SEQ ID NO: 4; and the sequence of the parental miR396i is shown in SEQ ID NO: 7, and the mutant miR396i undergoes deletions of 200th to 201st bases relative to the sequence shown in SEQ ID NO: 5.

In an embodiment, the sequence of the parental miR396a is shown in SEQ ID NO: 2, and the mutant miR396a undergoes a deletion of a 229th base relative to the sequence shown in SEQ ID NO: 2; the sequence of the parental miR396d is shown in SEQ ID NO: 5, and the mutant miR396d undergoes deletions of 19th to 29th bases relative to the sequence shown in SEQ ID NO: 5; and the sequence of the parental miR396f is shown in SEQ ID NO: 6, and the mutant miR396f undergoes a deletion of a 265th base relative to the sequence shown in SEQ ID NO: 6.

In an embodiment, the sequence of the parental miR396a is shown in SEQ ID NO: 2, and the mutant miR396a undergoes deletions of 229th to 233rd bases relative to the sequence shown in SEQ ID NO: 2; the sequence of the parental miR396b is shown in SEQ ID NO: 3, and the mutant miR396b undergoes a deletion of a 29th base relative to the sequence shown in SEQ ID NO: 3; the sequence of the parental miR396c is shown in SEQ ID NO: 4, and the mutant miR396c undergoes deletions of 229th to 230th bases relative to the sequence shown in SEQ ID NO: 4; and the sequence of the parental miR396d is shown in SEQ ID NO: 5, and the mutant miR396d undergoes deletions of 19th to 29th bases relative to the sequence shown in SEQ ID NO: 5.

In an embodiment, the sequence of the parental miR396a is shown in SEQ ID NO: 2, and the mutant miR396a undergoes deletions of 229th to 233rd bases relative to the sequence shown in SEQ ID NO: 2; the sequence of the parental miR396c is shown in SEQ ID NO: 4, and the mutant miR396c undergoes deletions of 228th to 253rd bases relative to the sequence shown in SEQ ID NO: 4; the sequence of the parental miR396f is shown in SEQ ID NO: 6, and the mutant miR396f undergoes a deletion of a 265th base relative to the sequence shown in SEQ ID NO: 6; and the sequence of the parental miR396i is shown in SEQ ID NO: 7, and the mutant miR396i undergoes deletions of 200th to 201st bases relative to the sequence shown in SEQ ID NO: 7.

In an embodiment, the sequence of the parental miR396b is shown in SEQ ID NO: 3, and the mutant miR396b undergoes deletions of 29th to 31st bases relative to the sequence shown in SEQ ID NO: 3; the sequence of the parental miR396c is shown in SEQ ID NO: 4, and the mutant miR396c undergoes deletions of 229th to 230th bases relative to the sequence shown in SEQ ID NO: 4; the sequence of the parental miR396d is shown in SEQ ID NO: 5, and the mutant miR396d undergoes deletions of 18th to 26th bases relative to the sequence shown in SEQ ID NO: 5; the sequence of the parental miR396f is shown in SEQ ID NO: 6, and the mutant miR396f undergoes deletions of 264th to 265th bases relative to the sequence shown in SEQ ID NO: 6; and the sequence of the parental miR396i is shown in SEQ ID NO: 7, and the mutant miR396i undergoes deletions of 200th to 201st bases relative to the sequence shown in SEQ ID NO: 7.

In another aspect, the present disclosure also provides a vector carrying the mutant miR396.

In an embodiment, the vector includes an expression vector, a shuttle vector, and an integration vector.

In an embodiment, the vector is a viral vector (such as a retroviral vector, a lentiviral vector, an adenoviral vector, an adeno-associated virus (AAV) vector, and a herpes simplex virus vector), and can also be a plasmid, a virus, a cosmid, a phage, etc., which are well known to those skilled in the art.

In an embodiment, the vector can further include at least one replication origin to achieve self-replication.

In a specific embodiment, the vector is a plasmid.

In an embodiment, the vector further includes an expression regulatory element.

In another aspect, the present disclosure provides a host cell carrying the mutant miR396 or the vector.

In an embodiment, the host cell is a eukaryotic cell, such as a yeast cell, an animal cell, or a plant cell.

In an embodiment, the plant includes an angiosperm and a gymnosperm.

In an embodiment, the plant includes a monocotyledonous plant and a dicotyledonous plant.

In an embodiment, the plant includes a herbaceous plant and a woody plant.

In an embodiment, the plant includes soybean, Arabidopsis thaliana, tobacco, rice, corn, sorghum, barley, wheat, millet, tomato, potato, quinoa, lettuce, rape, Brassica rapa, and strawberry.

In an embodiment, the host cell is a prokaryotic cell, such as Escherichia coli.

In another aspect, the present disclosure provides a gene editing reagent, where the gene editing reagent is able to cause generation of the mutant miR396 in soybean, the gene editing reagent includes Cas12i and guide RNA (gRNA); the gRNA includes a scaffold region for binding to the Cas12i and a guide sequence for hybridizing with a target sequence; and the gRNA targets miR396 in the soybean.

In an embodiment, an amino acid sequence of the Cas12i undergoes mutations of 369th and 433rd amino acids relative to a sequence shown in SEQ ID NO: 1.

In an embodiment, the Cas12i is produced by mutating a 369th amino acid into R and a 433rd amino acid into R based on the sequence shown in SEQ ID NO: 1.

In an embodiment, the amino acid sequence of the Cas12i undergoes mutations of 7th, 233rd, 267th, 369th, and 433rd amino acids relative to the sequence shown in SEQ ID NO: 1.

In an embodiment, the Cas12i is produced by mutating a 7th amino acid into R, a 233rd amino acid into R, a 267th amino acid into R, a 369th amino acid into R, and a 433rd amino acid into R based on the sequence shown in SEQ ID NO: 1.

In another aspect, the present disclosure provides a use of the mutant miR396, the vector, the host cell, or the gene editing reagent in preparation of soybean with an improved seed size, seed weight, number of seeds per plant, or yield; or in preparation of a reagent or kit for producing soybean with an improved seed size, seed weight, number of seeds per plant, or yield.

In another aspect, the present disclosure provides a method for producing soybean with an improved seed size, seed weight, number of seeds per plant, or yield, including the following steps:
(a) introducing the mutant miR396 into a soybean cell, a soybean seed, a soybean tissue, or a soybean part; and
(b) regenerating a soybean plant from a soybean cell, a soybean seed, a soybean tissue, or a soybean part produced in the step (a).

In an embodiment, the improved seed size means that a seed size of soybean carrying the mutant miR396 is at least 3%, preferably 5%, preferably 8%, preferably 10%, preferably 15%, preferably 20%, preferably 30%, preferably 40%, preferably 50%, preferably 60%, preferably 80%, and preferably 100% larger than a seed size of parental soybean (carrying WT miR396).

In an embodiment, the improved seed weight means that a single-seed weight, hundred-seed weight, or thousand-seed weight of soybean carrying the mutant miR396 is at least 5%, preferably 10%, preferably 12%, preferably 13%, preferably 15%, preferably 20%, preferably 25%, preferably 30%, preferably 40%, preferably 50%, and preferably 100% higher than a single-seed weight, hundred-seed weight, or thousand-seed weight of parental soybean (carrying WT miR396).

In an embodiment, the improved number of seeds per plant means that a number of seeds per plant of soybean carrying the mutant miR396 is at least 3%, preferably 5%, preferably 6%, preferably 10%, preferably 15%, preferably 20%, preferably 23%, preferably 25%, preferably 30%, preferably 40%, preferably 50%, and preferably 100% larger than a number of seeds per plant of parental soybean (carrying WT miR396).

In an embodiment, the improved yield means that a yield, per-plant yield, or per-mu yield of soybean carrying the mutant miR396 is at least 5%, preferably 10%, preferably 15%, preferably 20%, preferably 21%, preferably 25%, preferably 30%, preferably 40%, preferably 50%, preferably 60%, and preferably 100% higher than a yield, per-plant yield, or per-mu yield of parental soybean (carrying WT miR396).

In an embodiment, the introducing the mutant miR396 includes a step of mutating endogenous miR396 of soybean to introduce the mutant miR396.

In an embodiment, a mode for introducing the mutation includes natural variation, physical mutagenesis (such as ultraviolet-induced mutagenesis, X-ray-induced mutagenesis, or Y-ray-induced mutagenesis), chemical mutagenesis (such as nitrous acid, hydroxylamine, ethyl methanesulfonate (EMS), and nitrosoguanidine), biological mutagenesis (such as virus or bacterium-mediated mutagenesis), and gene editing.

In an embodiment, the mutant miR396 is introduced into the soybean cell, the soybean seed, the soybean tissue, or the soybean part through gene editing.

In an embodiment, the method includes the following steps:
(1) introducing an expression vector carrying a gene editing tool into a soybean cell, a soybean seed, a soybean tissue, or a soybean part,
(2) allowing the gene editing tool to act on endogenous miR396 of soybean to induce a mutation;
(3) selecting a mutant soybean cell, soybean seed, soybean tissue, soybean part, or soybean; and
(4) isolating the gene editing tool.

In an embodiment, the gene editing tool includes CRISPR, transcription activator-like effector nuclease (TALEN), and zinc-finger nuclease (ZEN).

In an embodiment, the gene editing tool is Cas12i.

In a specific embodiment, the gene editing is conducted with Cas12i in the soybean cell, the soybean seed, the soybean tissue, or the soybean part.

In an embodiment, an amino acid sequence of the Cas12i undergoes mutations of 369th and 433rd amino acids relative to a sequence shown in SEQ ID NO: 1.

In an embodiment, the Cas12i is produced by mutating a 369th amino acid into R and a 433rd amino acid into R based on the sequence shown in SEQ ID NO: 1.

In an embodiment, the amino acid sequence of the Cas12i undergoes mutations of 7th, 233rd, 267th, 369th, and 433rd amino acids relative to the sequence shown in SEQ ID NO: 1.

In an embodiment, the Cas12i is produced by mutating a 7th amino acid into R, a 233rd amino acid into R, a 267th amino acid into R, a 369th amino acid into R, and a 433rd amino acid into R based on the sequence shown in SEQ ID NO: 1.

In a specific embodiment, the Cas12i protein is linked to one or more nuclear localization signal (NLS) sequences. In an embodiment, the NLS sequence is linked to an N-terminus and/or a C-terminus of the protein.

In an embodiment, the introducing the mutant miR396 includes a step of allowing the mutant miR396 to express in the soybean cell, the soybean seed, the soybean tissue, the soybean part, or the soybean.

In an embodiment, the gene editing further includes a step of delivering the Cas12i into the soybean cell, the soybean seed, the soybean tissue, the soybean part, or the soybean.

In another aspect, the present disclosure also provides a method for gene editing of soybean, including the following steps:
(a) conducting the gene editing with Cas12i and gRNA in a soybean cell, a soybean seed, a soybean tissue, or a soybean part to produce a gene-edited soybean cell, soybean seed, soybean tissue, or soybean part, where the gRNA includes a scaffold region for binding to the Cas12i and a guide sequence for hybridizing with a target sequence, and the gRNA targets miR396 in soybean, and
(b) regenerating a soybean plant from the gene-edited soybean cell, soybean seed, soybean tissue, or soybean part obtained in the step (a).

In an embodiment, an amino acid sequence of the Cas12i undergoes mutations of 369th and 433rd amino acids relative to a sequence shown in SEQ ID NO: 1. Preferably, the 369th amino acid is mutated into arginine (Arg, R), and the 433rd amino acid is mutated into arginine (Arg, R).

In an embodiment, the amino acid sequence of the Cas12i undergoes mutations of 7th, 233rd, 267th, 369th, and 433rd amino acids relative to the sequence shown in SEQ ID NO: 1. Preferably, the 7th amino acid is mutated into arginine (Arg, R), the 233rd amino acid is mutated into arginine (Arg, R), the 267th amino acid is mutated into arginine (Arg, R), the 369th amino acid is mutated into arginine (Arg, R), and the 433rd amino acid is mutated into arginine (Arg, R).

In a specific embodiment, the Cas12i is mutant Cas12i (or optimized Cas12i), which is a mutant protein produced through mutations of 369th and 433rd amino acids (both are mutated into R) or a mutant protein produced through mutations of 7th, 233rd, 267th, 369th, and 433rd amino acids (all are mutated into R) based on SEQ ID NO: 1. The mutant Cas12i protein exhibits a significantly-improved editing activity compared with the WT Cas12i shown in SEQ ID NO: 1.

In an embodiment, the gRNA includes a first segment and a second segment. The first segment is also referred to as “scaffold region”, “protein binding segment”, “protein binding sequence”, or “direct repeat (DR)”. The second segment is also referred to as “nucleic acid-targeted targeting sequence”, “nucleic acid-targeted targeting segment”, or “target sequence-targeted guide sequence”.

The “first segment”, “scaffold region”, “protein binding segment”, “protein binding sequence”, or “DR” of the gRNA can interact with the Cas12i protein of the present disclosure to produce a complex of the Cas12i protein and the gRNA. The gRNA of the present disclosure guides the Cas12i protein interacting therewith to a specific nucleotide sequence in a target nucleic acid under an action of the nucleic acid-targeted targeting sequence.

The nucleic acid-targeted targeting sequence or the nucleic acid-targeted targeting segment of the present disclosure includes a nucleotide sequence complementary to a sequence in the target nucleic acid. In other words, the nucleic acid-targeted targeting sequence or the nucleic acid-targeted targeting segment of the present disclosure can interact with the target nucleic acid in a sequence-specific manner through hybridization (namely, base pairing). Therefore, the nucleic acid-targeted targeting sequence or the nucleic acid-targeted targeting segment can be changed or can be modified to hybridize with any desired sequence in the target nucleic acid.

Preferably, the gRNA includes a first segment and a second segment in a direction from S′ terminus to 3′ terminus.

In the present disclosure, the second segment can also be understood as a guide sequence to hybridize with a target sequence.

In a specific embodiment, a target sequence-targeted guide sequence of the gRNA is shown in any one of SEQ ID NOS: 8-11.

In a specific embodiment, DR of the gRNA is shown in SEQ ID NO: 12.

In other embodiments, the DR of the gRNA can also be produced through a base deletion, substitution, or addition based on SEQ ID NO: 12, as long as the binding ability to Cas12i can be guaranteed, such as “agagaaugugugcauagucaacac” (SEQ ID NO: 13), “agagaaugugugcauagucuacac” (SEQ ID NO: 14), “agagaaugugugcauaguccacac” (SEQ ID NO: 15), or “agagaaugugugcauagucgacac” (SEQ ID NO: 16) recorded in the Chinese patent application (CN113337502A).

In an embodiment, a nucleic acid sequence encoding the Cas12i protein and a nucleic acid encoding the gRNA are artificially synthesized.

In an embodiment, the method for gene editing in the present disclosure includes a step of delivering the Cas12i and the gRNA into the soybean cell, the soybean seed, the soybean tissue, the soybean part, or the soybean.

The delivering can be conducted by any method known in the art. Such a method includes, but is not limited to, transformation, transfection, electroporation, lipofection, microinjection, sonoporation, gene gun, calcium phosphate-mediated transfection, cationic transfection, lipofectin transfection, dendritic transfection, heat-shock transfection, nucleofection, magnetofection, puncture transfection, optical transfection, reagent-enhanced nucleic acid intake, and delivery via a liposome, an immunoliposome, a viral particle, a vector, a viral vector, an artificial virus, etc.

In some embodiments, one or more AAV vectors, lentiviral vectors, nanoparticles, or a combination thereof are used to deliver one or more components of the Cas12i and the gRNA.

In an embodiment, the Cas12i and the gRNA are delivered into the soybean cell, the soybean seed, the soybean tissue, the soybean part, or the soybean through Agrobacterium-mediated transformation.

The gRNA targets miR396 in the soybean cell, and guides the Cas12i protein to reach a specified genomic locus to modify, edit, or cleave a target sequence, thereby mutating the miR396.

In an embodiment, the method includes a step of introducing the mutant miR396 into the soybean cell, the soybean seed, the soybean tissue, or the soybean part.

In another aspect, the present disclosure also provides a method for producing soybean with an improved seed size, seed weight, number of seeds per plant, or yield, including a step of hybridizing a soybean seed or a soybean plant prepared by the method with another soybean variety to produce the soybean with the improved seed size, seed weight, number of seeds per plant, or yield.

### Terms and Definitions

Unless defined otherwise, the technical and scientific terms used herein have the same meanings as commonly understood by those of ordinary skill in the art.

The terms “polynucleotide”, “nucleotide sequence”, “nucleic acid sequence”, “nucleic acid molecule”, and “nucleic acid” can be used interchangeably, and include DNA, RNA, or a hybrid thereof, which may be double-stranded or single-stranded.

The terms “protein”, “polypeptide”, and “peptide” can be used interchangeably in the present disclosure, and refer to a polymer of amino acid residues, including a polymer in which one or more amino acid residues are a chemical analog of a natural amino acid residue. The protein and polypeptide of the present disclosure can be produced through recombination or chemical synthesis. The term “mutant protein” or “mutated protein” refers to a protein that is produced through the substitution, insertion, deletion, and/or addition of one or more amino acid residues based on an amino acid sequence of a parent protein.

The term “amino acid” refers to a carboxylic acid with amino. Various proteins in organisms each are composed of 20 essential amino acids.

The term “encoding” refers to an inherent characteristic of a specific nucleotide sequence in a polynucleotide, such as a gene cDNA or mRNA, which serves as a template for the synthesis of a defined nucleotide sequence (namely, rRNA, (RNA, and mRNA) or a defined amino acid sequence and the synthesis of other polymers and macromolecules in a biological process of a biological characteristic thereof. Therefore, if the transcription and translation of mRNA corresponding to a gene produces a protein in a cell or another biological system, the gene encodes the protein.

As used herein, the term “identity” refers to the sequence matching between two polypeptides or between two nucleic acids. When specified positions in two sequences to be compared are occupied by the same base or amino acid monomer subunit (for example, a specified position in each of two DNA molecules is occupied by adenine, or a specified position in each of two polypeptides is occupied by lysine), the molecules are identical at the position. The “percent identity” between two sequences is a function of a number of matched positions shared by the two sequences/a number of compared positions×100. For example, if 6 of 10 positions in one of two sequences match corresponding positions in the other one of the two sequences, then the two sequences have an identity of 60%. For example, DNA sequences CTGACT and CAGGTT have an identity of 50% (3 of 6 positions are matching). Generally, the comparison is conducted by aligning two sequences to produce the maximum identity. Such alignment can be achieved by using, for example, a method of Needleman et al. (1970) J. Mol. Biol. 48:443-453 that can be conveniently implemented by a computer program such as Align program (DNAstar, Inc.). The percent identity between two amino acid sequences can also be determined with an algorithm of E. Meyers and W. Miller (Comput. Appl Biosci., 4:11-17 (1988) integrated into the ALIGN program (version 2.0), a PAM120 weight residue table, a gap length penalty of 12, and a gap penalty of 4. In addition, the percent identity between two amino acid sequences can be determined by using an algorithm of Needleman and Wunsch (J Mol Biol. 48:444-453 (1970)) in the GAP program integrated into the GCG software package (available on www.gcg.com), a Blossum 62 matrix or a PAM250 matrix, a gap weight of 16, 14, 12, 10, 8, 6, or 4, and a length weight of 1, 2, 3, 4, 5, or 6.

As used herein, the term “regulatory element”, also known as “regulation element”, is intended to include a promoter, a terminator sequence, a leader sequence, a polyadenylation sequence, a signal peptide coding region, a marker gene, an enhancer, an internal ribosome entry site (IRES), and other expression control elements (for example, a transcriptional termination signal, such as a polyadenylation signal and a poly-U sequence), and the detailed description can be seen in Goeddel, “GENE EXPRESSION TECHNOLOGY: METHODS IN ENZYMOLOGY” 185, Academic Press, San Diego, California (1990). In some cases, a regulatory element includes sequences that guide the constitutive expression of a nucleotide sequence in many types of host cells and sequences that guide the expression of the nucleotide sequence only in some host cells (such as a tissue-specific regulatory sequence). A tissue-specific promoter can mainly guide the expression in a desired tissue of interest, such as muscles, neurons, bones, skin, blood, specific organs (such as liver and pancreas), or specific cell types (such as lymphocytes). In some cases, a regulatory element can also guide the expression in a time-dependent manner (such as in a cell cycle-dependent or developmental stage-dependent manner), which may be or may not be tissue or cell type-specific. In some cases, the term “regulatory element” covers enhancer elements, such as WPRE; CMV enhancer; R-US' fragment in LTR of HTLV-I ((Mol. Cell. Biol., Vol 8 (1): 466-472, 1988); SV40 enhancer; and an intron sequence between exons 2 and 3 of rabbit β-globin (Proc. Natl. Acad. Sci. USA., Vol. 78 (3): 1527-31, 1981).

As used herein, the term “promoter” has the meaning well known to those skilled in the art, and refers to a non-coding nucleotide sequence that is located upstream of a gene and can promote the expression of a downstream gene. A constitutive promoter is a nucleotide sequence that will result in the generation of a gene product in a cell under most or all physiological conditions of the cell after the promoter is operably linked to a polynucleotide encoding or defining the gene product. An inducible promoter is a nucleotide sequence that will cause the generation of a gene product in a cell only when there is an inducer corresponding to the promoter in the cell after the promoter is operably linked to a polynucleotide encoding or defining the gene product. A tissue-specific promoter is a nucleotide sequence that will cause the generation of a gene product in a cell basically only when the cell is a cell of the tissue type corresponding to the promoter after the promoter is operably linked to a polynucleotide encoding or defining the gene product.

The term “nuclear localization signal” or “nuclear localization sequence” (NLS) is an amino acid sequence that tags a protein for import into a nucleus through nuclear transport, that is, a protein with NLS is transported into a nucleus. Typically, NLS includes positively-charged Lys or Arg residues that are exposed on a surface of a protein. Exemplary NLS includes, but is not limited to, SV40 large T antigen, EGL-13, c-Myc, and TUS protein.

As used herein, the term “operably linked” means that a nucleotide sequence of interest is linked to one or more regulatory elements in a manner allowing the expression of the nucleotide sequence (for example, in an in vitro transcription/translation system or in a host cell when the vector is introduced into the host cell).

### WT

As used herein, the term “WT” has the meaning commonly understood by those skilled in the art, and indicates a typical form of an organism, a strain, or a gene, or a characteristic to distinguish the organism, strain, or gene in nature from a mutant or variant form thereof, which can be isolated from a natural source and is not artificially modified intentionally.

### Vector

The term “vector” refers to a nucleic acid that can deliver another nucleic acid linked thereto. A vector includes, but is not limited to, a single-stranded, double-stranded, or partially double-stranded nucleic acid; a nucleic acid with one or more free ends or without free ends (such as circular); DNA, RNA, or a nucleic acid of both; and other diverse polynucleotides known in the art. A vector can be introduced into a host cell through transformation, transduction, or transfection, such that a genetic material element carried by the vector can be expressed in the host cell. A vector can be introduced into a host cell to produce a transcript, a protein, or a peptide, including the protein, the fusion protein, the isolated nucleic acid, etc. (for example, a CRISPR transcript, such as a nucleic acid transcript, a protein, or an enzyme) described herein. A vector may include a variety of elements to control the expression, including, but not limited to, a promoter sequence, a transcription initiation sequence, an enhancer sequence, a selection element, and a reporter gene. A vector may also include a replication origin.

A type of a vector is a plasmid, which refers to a circular double-stranded DNA loop where an additional DNA fragment can be inserted, for example, by a standard molecular cloning technique.

Another type of a vector is a viral vector in which a virus-derived DNA or RNA sequence is present in a vector for packaging a virus (such as a retrovirus, a replication-defective retrovirus, an adenovirus, a replication-defective adenovirus, and AAV). A viral vector also includes a polynucleotide carried by a virus for transfection into a host cell. Some vectors (for example, bacterial vectors with bacterial replication origins and episomal mammalian vectors) can autonomously replicate in a host cell into which these vectors are introduced.

Other vectors (such as non-episomal mammalian vectors) will be integrated into a genome of a host cell after being introduced into the host cell, and thus will replicate with the genome of the host cell. Moreover, some vectors can guide the expression of genes operably linked thereto. Such vectors are referred to as expression vectors herein.

### Host Cell

As used herein, the term “host cell” refers to a cell that can be introduced with a vector, including, but not limited to, a prokaryotic cell such as Escherichia coli or Bacillus subtilis, and a eukaryotic cell such as a microbial cell, a fungal cell, an animal cell, and a plant cell.

Those skilled in the art will understand that the design of an expression vector may depend on factors such as the selection of a host cell to be transformed, and a desired expression level.

### Animal

For example, the animal may be a mammal, such as bovine, equine, sheep, swine, canine, feline, leporid, rodent (such as mouse or rat), non-human primate (such as macaque or cynomolgus monkey), or human. In some embodiments, the subject (such as human) suffers from a disorder (such as a disorder caused by a disease-associated gene defect).

### Plant

The term “plant” should be understood as any differentiated multicellular organism capable of conducting photosynthesis, including: any crop plants at a mature or developmental stage, especially monocotyledonous or dicotyledonous plants; vegetable crops including artichoke, turnip cabbage, arugula, leek, asparagus, lettuce (such as head lettuce, leaf lettuce, and romaine lettuce), bok choy, malanga, melons (such as cantaloupe, watermelon, crenshaw melon, honeydew melon, and Cantaloupe), rape crops (such as Brussels sprout, cabbage, cauliflower, broccoli, borecole, kale, Chinese cabbage, and bok choy), cardoon, carrot, napa, okra, onion, celery, parsley, chickpea, parsnip, chicory, pepper, potato, gourd (such as marrow squash, cucumber, zucchini, cushaw, and pumpkin), radish, dried ball onion, rutabaga, purple eggplant (also known as eggplant), salsify, lettuce, shallot, endive, garlic, spinach, green onion, cushaw, greens, Beta vulgaris (sugar beets and fodder beets), sweet potato, Swiss chard, wasabi, tomato, turnip, and spices; fruits and/or vine crops such as apple, apricot, cherry, nectarine, peach, pear, plum, prune, cherry, quince, almond, chestnut, hazelnut, pecan, pistachio, walnut, citrus, blueberry, boysenberry, cranberry, currant, loganberry, raspberry, strawberry, blackberry, grape, avocado, banana, kiwi, persimmon, pomegranate, pineapple, tropical fruit, pome, melon, mango, papaya, and lychee; field crops, such as clover, Medicago, evening primrose, meadowfoam, corn/maize (forage maize, sweet corn, and popcorn), lupulus, jojoba, peanut, rice, safflower, small grain crops (barley, oat, rye, wheat, etc.), sorghum, tobacco, kapok, Fabaceae plants (beans, lentil, pea, and soybean), oil plants (rape, leaf mustard, poppy, olive, sunflower, coconut, castor oil plant, cocoa bean, and groundnut), Arabidopsis, fiber plants (cotton, flax, hemp, and jute), Lauraceae (cinnamon and camphor), or a plant such as coffee, sugar cane, tea, and natural rubber plants; and/or bedding plants such as a flowering plant, cactus, a succulent plant, and/or an ornamental plant, and trees such as forests (broad-leaved and evergreen trees, such as conifers), fruit trees, ornamental trees, nut-bearing trees, shrubs, and other seedlings.

The term “plant tissue” or “plant part” includes a plant cell, a protoplast, a plant tissue culture, a plant callus, a plant piece, a plant embryo, a pollen, an ovule, a seed, a leaf, a stem, a flower, a branch, a seedling, a fruit, a kernel, a spike, a root, a root tip, an anther, etc.

The term “plant cell” should be understood as any cell derived from or found in a plant, and can produce, for example, undifferentiated tissues such as calli, differentiated tissues such as embryos, constituent parts of a plant, plants, or seeds.

### Gene Editing

The gene editing technology includes CRISPR technology, TALEN technology, and ZEN technology. The CRISPR technology refers to clustered regularly interspaced short palindromic repeats coming from the immune system of microorganisms. A gene editing tool includes gRNA and a Cas protein (such as Cas9, Cpf1, Cas12b, and Cas12i). A gene editing tool of TALEN refers to a restriction enzyme capable of cleaving a specific DNA sequence, which includes a TAL effector DNA binding domain and a DNA cleavage domain. A gene editing tool of ZEN refers to a restriction enzyme capable of cleaving a specific DNA sequence, which includes a zinc-finger DNA binding domain and a DNA cleavage domain. It is well known to those skilled in the art that an intracellular genome can be edited by constructing nucleotides encoding a gene editing tool and other regulatory elements into a suitable vector and then transforming the vector into a cell, where a type of the editing includes gene knockout, insertion, and base editing.

### CRISPR System

As used herein, the terms “CRISPR-Cas system” and “CRISPR system” can be used interchangeably and have the meaning commonly understood by those skilled in the art. The CRISPR-Cas system or the CRISPR system usually includes a transcription product or other elements related to the expression of a Cas gene, or a transcription product or other elements capable of guiding the activity of the Cas gene.

### Cas Protein

A Cas protein or CRISPR-associated protein refers to a nuclease suitable for a CRISPR system. Preferably, the Cas protein is a CRISPR enzyme, including, but not limited to: a Cas9 protein, a Cas12 protein, a Cas13 protein, a Cas14 protein, a Csm1 protein, and an FDK1 protein. Cas proteins can exhibit varying structures depending on their origins, such as SpCas9 derived from Streptococcus pyogenes and SaCas9 derived from Staphylococcus aureus. Cas proteins can also be classified based on structural characteristics (such as domains). For example, the Cas12 family may include Cas12a (also known as Cpf1), Cas12b, Cas12c, Cas12i, etc. The Cas protein may be double-stranded or single-stranded or may have no cleavage activity. The Cas protein of the present disclosure may be WT or a mutant thereof. A mutation type of the mutant may include amino acid substitution, substitution, or deletion. The mutant may or may not change an enzyme cleavage activity of the Cas protein. Those skilled in the art know that a variety of Cas proteins with nucleic acid cleavage activities have been reported in the art. The known proteins or modified variants thereof all can achieve the function of the present disclosure, which is incorporated into the protection scope of the present disclosure by reference.

It is clear to those skilled in the art that a structure of a protein can be changed without adversely affecting the activity and functionality of the protein. For example, one or more conservative amino acid substitutions can be introduced into an amino acid sequence of a protein without adversely affecting the activity and/or three-dimensional structure of the protein molecule. Those skilled in the art are aware of examples and implementations of the conservative amino acid substitutions. Specifically, an amino acid residue can be substituted by another amino acid residue that belongs to the same group as the amino acid residue to be substituted. That is, a nonpolar amino acid residue can be substituted by another nonpolar amino acid residue; an uncharged polar amino acid residue can be substituted by another uncharged polar amino acid residue; a basic amino acid residue can be substituted by another basic amino acid residue; and an acidic amino acid residue can be substituted by another acidic amino acid residue. Such substituted amino acid residues may be or may not be encoded by genetic codes. As long as a substitution does not result in a loss of a biological activity of a protein, a conservative substitution in which an amino acid is substituted by another amino acid belonging to the same group falls within the scope of the present disclosure. Therefore, an amino acid sequence of the protein of the present disclosure may include one or more conservative substitutions, and these conservative substitutions may be preferably generated according to Table 1. In addition, the present disclosure covers a protein with one or more other non-conservative substitutions, as long as the non-conservative substitutions do not significantly affect the desired function and biological activity of the protein of the present disclosure.

Conservative amino acid substitutions can be made at one or more predicted non-essential amino acid residues. Non-essential amino acid residues are amino acid residues that can be changed (deleted or substituted) without changing a biological activity, while essential amino acid residues are required for a biological activity. A conservative amino acid substitution refers to a substitution in which an amino acid residue is substituted by an amino acid residue with a similar side chain. An amino acid substitution can be made in a non-conservative region of the Cas mutant protein. Generally, such a substitution is not made to a conservative amino acid residue or an amino acid residue located within a conservative motif because such a residue is required for an activity of the protein. However, those skilled in the art should understand that a functional variant may have few conservative or non-conservative variations in conservative regions.

TABLE 1

| Initial residue | Representative substitution | Preferred substitution |
|---|---|---|
| Ala(A) | Val; Leu; Ile | Val |
| Arg(R) | Lys; Gln; Asn | Lys |
| Asn(N) | Gln; His; Lys; Arg | Gln |
| Asp(D) | Glu | Glu |
| Cys(C) | Ser | Ser |
| Gln(Q) | Asn | Asn |
| Glu(E) | Asp | Asp |
| Gly(G) | Pro; Ala | Ala |
| His(H) | Asn; Gln; Lys; Arg | Arg |
| Ile(I) | Leu; Val; Met; Ala; Phe | Leu |
| Leu(L) | Ile; Val; Met; Ala; Phe | Ile |
| Lys(K) | Arg; Gln; Asn | Arg |
| Met(M) | Leu; Phe; Ile | Leu |
| Phe(F) | Leu; Val; Ile; Ala; Tyr | Leu |
| Pro(P) | Ala | Ala |
| Ser(S) | Thr | Thr |
| Thr(T) | Ser | Ser |
| Trp(W) | Tyr; Phe | Tyr |
| Tyr(Y) | Trp; Phe; Thr; Ser | Phe |
| Val(V) | Ile; Leu; Met; Phe; Ala | Leu |

It is well known in the art that one or more amino acid residues can be changed (substituted, deleted, truncated, or inserted) at an N-terminus and/or a C-terminus of a protein with a functional activity of the protein still retained. Therefore, a protein produced by changing one or more amino acid residues at an N-terminus and/or a C-terminus of the Cas protein while retaining a desired functional activity also falls within the scope of the present disclosure. Such a change may include a change introduced by a modern molecular method such as polymerase chain reaction (PCR), and the method includes PCR amplification that alters or extends a protein coding sequence by introducing an amino acid coding sequence into an oligonucleotide used for the PCR amplification.

It should be recognized that a protein can be altered in various ways, including amino acid substitution, deletion, truncation, and insertion, and methods for such operations are generally known in the art. For example, amino acid sequence variants of the protein can be prepared through mutations of DNA. It can also be completed through other mutagenesis forms and/or through directed evolution. For example, the known mutagenesis, recombination, and/or shuffling methods can be used in combination with the related screening method to achieve the substitution, deletion, and/or insertion of one or more amino acids.

Those skilled in the art can understand that these small amino acid changes in the Cas protein of the present disclosure can be naturally present (for example, natural mutations) or can be induced (for example, using r-DNA technology) without affecting a function or activity of the protein. If these mutations occur in a catalytic domain, an active site, or other functional domains of the protein, the properties of the polypeptide may be changed, but the polypeptide may retain its activity. If the present mutations are not close to a catalytic domain, an active site, or other functional domains, it can be expected that there is a minimal impact.

Those skilled in the art can identify the essential amino acids of the Cas mutant protein of the present disclosure according to a method known in the art, such as site-directed mutagenesis or protein evolution or bioinformatics analysis. The catalytic domain, active site, or other functional domains of the protein can also be determined through the physical analysis of a structure. For example, it can be determined by a technique such as nuclear magnetic resonance, crystallography, electron diffraction, or photoaffinity labeling in combination with a putative amino acid mutation at a key site.

In the present disclosure, an amino acid residue can be represented by a single letter or three letters, such as: alanine (Ala, A), valine (Val, V), glycine (Gly, G), leucine (Leu, L), glutamine (Gln, Q), phenylalanine (Phe, F), tryptophan (Trp, W), tyrosine (Tyr, Y), aspartic acid (Asp, D), asparagine (Asn, N), glutamic acid (Glu, E), lysine (Lys, K), methionine (Met, M), serine (Ser, S), threonine (Thr, T), cysteine (Cys, C), proline (Pro, P), isoleucine (Ile, I), histidine (His, H), and arginine (Arg, R).

The term “AxxB” means that an amino acid A at position xx is mutated into an amino acid B, that is, the amino acid A at the position xx from an N-terminus is mutated into the amino acid B, unless otherwise specified. For example, N369R indicates that N at position 369 is mutated into R. When there are mutations simultaneously at a plurality of amino acid sites, the mutations can be expressed in a form such as N369R, S433R, or N369R/S433R. For example, N369R and S433R mean that 369th N is mutated into R and 433rd N is mutated into R.

A specific amino acid position (number) in the protein of the present disclosure is determined by aligning an amino acid sequence of the target protein with SEQ ID NO: 1 using a standard sequence alignment tool. For example, the Smith-Waterman algorithm or the CLUSTALW2 algorithm is used to align two sequences, and the sequences are considered aligned when an alignment score is the highest. The alignment score can be calculated according to the method described in Wilbur, W. J. and Lipman, D. J. (1983) Rapid similarity searches of nucleic acid and protein data banks. Proc. Natl. Acad. Sci. USA, 80:726-730. In the ClustalW2 (1.82) algorithm, default parameters are preferably adopted: protein gap opening penalty=10.0; protein gap extension penalty=0.2; protein matrix=Gonnet, protein/DNA end gap=−1; and protein/DNAGAPDIST=4. The AlignX program (a part of the vectorNTI group) may preferably be used to align an amino acid sequence of the protein of the present disclosure with SEQ ID NO: 1 under default parameters suitable for multiple alignment (gap opening penalty: 10; and gap extension penalty: 0.05) to determine a position of a specific amino acid in the protein.

Those skilled in the art can use the common software in the art, such as Clustal Omega, to compare and align an amino acid sequence of any parental Cas protein with SEQ ID NO: 1 for sequence identity, so as to obtain an amino acid site in the parental Cas protein that corresponds to an amino acid site defined based on SEQ ID NO: 1 in the present application.

A biological function of the Cas protein includes, but is not limited to, an activity to bind to gRNA, an endonuclease activity, and an activity to bind to a specific site of a target sequence and cleave the target sequence under the guidance of gRNA, including, but not limited to, Cis cleavage activity and Trans cleavage activity.

In the present disclosure, the “Cas mutant protein” can also be referred to as a mutated Cas protein, an optimized Cas protein, or a Cas protein variant. ØRNA

As used herein, the terms “gRNA”, “mature crRNA”, and “guide sequence” can be used interchangeably and have the meaning commonly understood by those skilled in the art. Generally, gRNA can include DR and a guide sequence, or is essentially composed of or is composed of a DR and a guide sequence.

In some cases, the guide sequence can be any polynucleotide sequence that shows sufficient complementarity with a target sequence to hybridize with the target sequence and guide the specific binding of a CRISPR/Cas complex to the target sequence. In an embodiment, under the optimal alignment, a complementarity degree between a guide sequence and a corresponding target sequence is at least 50%, at least 60%, at least 70%, at least 80%, at least 90%, at least 95%, or at least 99%. Determining the optimal alignment is within the competence of those of ordinary skill in the art. For example, there are published and commercially available alignment algorithms and programs, including, but not limited to, Smith-Waterman, Bowtie, Geneious, Biopython, and SeqMan in ClustalW and matlab.

### Target Sequence

A target sequence refers to a polynucleotide targeted by a guide sequence in gRNA, such as a sequence complementary to the guide sequence. The hybridization between a target sequence and a guide sequence will promote the generation of a CRISPR/Cas complex (including a Cas protein and gRNA). Complete complementarity is not necessary, as long as there is sufficient complementarity to cause hybridization and promote the generation of a CRISPR/Cas complex.

A target sequence can include any polynucleotide, such as DNA or RNA. In some cases, a target sequence is located inside or outside a cell. In some cases, a target sequence is located in a nucleus or cytoplasm of a cell. In some cases, a target sequence may be located in an organelle of a eukaryotic cell, such as a mitochondrion or a chloroplast. A sequence or a template that can be recombined into a target gene locus with a target sequence is called “edit template” or “edit polynucleotide” or “edit sequence”. In an embodiment, the edit template is an exogenous nucleic acid. In an embodiment, the recombination refers to homologous recombination.

In the present disclosure, the “target sequence” or “target polynucleotide” or “target nucleic acid” can be any endogenous or exogenous polynucleotide for a cell (such as a eukaryotic cell). For example, the target polynucleotide may be a polynucleotide present in a nucleus of a eukaryotic cell. The target polynucleotide may be a sequence encoding a gene product (such as a protein) or a non-coding sequence (such as a regulatory polynucleotide or useless DNA), In some cases, the target sequence should be related to a protospacer adjacent motif (PAM).

miR396

miR396 is a highly-conserved endogenous non-coding single-stranded small RNA molecule with a length of 20 to 24 nucleotides. The plant miR396 family undergoes minimal sequence variation over long-term biological evolution, and is highly conserved. miR396 can bind to mRNA of GRF for a target gene through base pairing to regulate the expression of GRF, thereby affecting the growth and development of various organs of a plant.

Various miR396s (miR396a/b/c/d/e/f/g/h/i/j/k) have been found in soybean. These miR396s are identified from sequencing data. Functions of these miR396s have not been verified by related studies.

In an embodiment, a sequence of soybean miR396a is shown in SEQ ID NO: 2, a sequence of soybean miR396b is shown in SEQ ID NO: 3, a sequence of soybean miR396c is shown in SEQ ID NO: 4, a sequence of soybean miR396d is shown in SEQ ID NO: 5, a sequence of soybean miR396f is shown in SEQ ID NO: 6, and a sequence of soybean miR396i is shown in SEQ ID NO: 7.

The sequences involved in the present disclosure are as follows:

| Sequence No. (SEQ ID NO:) | Sequence description |
|---|---|
| 1 | Amino acid sequence of WT Cas12i |
| 2 | Nucleotide sequence of GmmiR396a |
| 3 | Nucleotide sequence of GmmiR396b |
| 4 | Nucleotide sequence of GmmiR396c |
| 5 | Nucleotide sequence of GmmiR396d |
| 6 | Nucleotide sequence of GmmiR396f |
| 7 | Nucleotide sequence of GmmiR396i |
| 8 | Guide sequence 1 of gRNA |
| 9 | Guide sequence 2 of gRNA |
| 10 | Guide sequence 3 of gRNA |
| 11 | Guide sequence 4 of gRNA |
| 12 | DR sequence of gRNA |

### Major Advantages of the Present Disclosure

In the present disclosure, miR396 is subjected to targeted editing by the Cas12i gene editing technology. It has been discovered that, when miR396a, miR396b, miR396c, miR396d, miR396f, or miR396i in soybean undergoes a base mutation, the soybean has an improved seed size and yield.

### BRIEF DESCRIPTION OF THE DRAWINGS

FIG. 1 shows the validation of editing efficiencies of Cas12i proteins, where 1 is for WT Cas12i, and 2 is for a Cas12i protein with a combined mutation of N369R and S433R;

FIG. 2A shows the comparison of seed sizes between an edited plant acdf and WT soybean (Zhonghuang 302, WT), FIG. 2B shows the comparison of seed sizes between an edited plant abcdf and WT soybean (Zhonghuang 302, WT), FIG. 2C shows the comparison of seed sizes between an edited plant acd and WT soybean (Zhonghuang 302, ZH302), and FIG. 2D shows the comparison of seed sizes between an edited plant cdf and WT soybean (Zhonghuang 302, ZH302);

FIG. 3A shows the comparison of seed lengths of seeds of an edited plant acd and an edited plant cdf with WT soybean (Zhonghuang 302, ZH302), FIG. 3B shows the comparison of seed widths of seeds of an edited plant acd and an edited plant cdf with WT soybean (Zhonghuang 302, ZH302), and FIG. 3C shows the comparison of seed thicknesses of seeds of an edited plant acd and an edited plant cdf with WT soybean (Zhonghuang 302, ZH302);

FIG. 4A shows the comparison of seed sizes between an edited plant aci and WT soybean (Zhonghuang 302, ZH302), FIG. 4B shows the comparison of seed sizes between an edited plant adf and WT soybean (Zhonghuang 302, ZH302), FIG. 4C shows the comparison of seed sizes between an edited plant acbd and WT soybean (Zhonghuang 302, ZH302), FIG. 4D shows the comparison of seed sizes between an edited plant acfi and WT soybean (Zhonghuang 302, ZH302), and FIG. 4E shows the comparison of seed sizes between an edited plant bedfi and WT soybean (Zhonghuang 302, ZH302); and

FIG. 5A shows the comparison of seed lengths of seeds of edited plants aci, adf, abcd, acfi, and bcdfi with WT soybean (Zhonghuang 302, ZH302), FIG. 5B shows the comparison of seed widths of seeds of edited plants aci, adf, abcd, acfi, and bedfi with WT soybean (Zhonghuang 302, ZH302), and FIG. 5C shows the comparison of seed thicknesses of seeds of edited plants aci, adf, abcd, acfi, and bodfi with WT soybean (Zhonghuang 302, ZH302).

### DETAILED DESCRIPTION OF THE EMBODIMENTS

The present disclosure will be further explained below in conjunction with an example. The following example is only a preferred example of the present disclosure, and is not intended to limit the present disclosure in other forms. Any technical personnel familiar with the profession may use the technical content disclosed above to derive equivalent examples through equivalent changes. Any simple modification or equivalent change made to the following example according to the technical essence of the present disclosure without departing from the content of the solutions of the present disclosure shall fall within the protection scope of the present disclosure.

### Example 1 Acquisition of Cas12i (N369R and S433R) and Cas12i (S7R. D233R, D267R, N369R, and S433R) Mutant Proteins and Validation of Editing Activities Thereof

The applicants conducted site-directed mutagenesis on the potential amino acids in Cas1213 for binding to a target sequence by a bioinformatics method. An amino acid sequence of WT Cas12i was shown in SEQ ID NO: 1. A method for the site-directed mutagenesis was a common method in the art. In this example, variants of the Cas protein were produced through PCR-based site-directed mutagenesis. A specific method was as follows: A DNA sequence for the Cas1213 protein was designed as two parts with a mutation site as a center, and two pairs of primers were designed to amplify the two parts of the DNA sequence, respectively. A sequence to be mutated was introduced into the primers. Finally, two amplified fragments were loaded on a pcDNA3.3-eGFP vector through Gibson cloning. A combination of mutations was constructed by splitting the DNA for the Cas1213 protein into a plurality of fragments and then conducting PCR and Gibson cloning. A fragment amplification kit was Trans Start Fast Pfu DNA Polymerase (including 2.5 mM dNTPs), and a specific experimental procedure was detailed in instructions. A gel recovery kit was FastPure® Gel DNA Extraction Mini Kit, and a specific experimental procedure was detailed in instructions. A kit for vector construction was pEASY-Basic Seamless Cloning and Assembly Kit (CU201-03), and a specific experimental procedure was detailed in instructions. The mutant amino acid sites involved in this example and the primer sequences adopted accordingly were listed in the table below:

| | | Amino acid |
|---|---|---|
| | | mutation type |
| | | (amino acid |
| | | site from an |
| | | N-terminus in |
| Primer | | SEQ ID |
| name | Primer sequence | NO: 1) |
| i3-H6-F | CGGCTTTAACAGGAAGTACAA | N369R |
| | CGTGGTCGCCGAGCTGTACAA | |
| | SEQ ID NO: 17) | |
| i3-H6-R | TTGTACTTCCTGTTAAAGCCG | |
| | ATGTGCTCTGGCTTGATCA | |
| | (SEQ ID NO: 18) | |
| i3-H9-F | CGGTTTAACCAGCGGGAGGAGA | S433R |
| | AGCTGCTGOGGATCAAG (SEQ | |
| | ID NO: 19) | |
| i3-H9-R | TCCTCCCGCTGGTTAAACCGGG | |
| | CCACCCTGCCCCACTTC | |
| | (SEQ ID NO: 20) | |

In this example, a Cas12i mutant protein with both mutations of N369R and S433R was produced through site-directed mutagenesis. Compared to SEQ ID NO: 1, in an amino acid sequence of the mutant protein, N at position 369 was mutated into R and S at position 433 was mutated into R.

To verify a gene editing activity of the Cas12i mutant protein in an animal cell, the following target was designed for the FUT8 gene in Chinese hamster ovary (CHO) cell:

FUT8-Cas-XX-g3:
(SEQ ID NO: 21)
TTCCAGCCAAGGTTGTGGACGGATCA,

where an italicized region was a PAM sequence and an underlined region was a targeting region. A vector pcDNA3.3 was modified to carry an enhanced green-fluorescent protein (EGFP) and a PuroR resistance gene. A fusion protein SV40 NLS-Cas-XX was inserted through enzymatic cleavage sites XbaI and PstI, and a U6 promoter and a gRNA sequence were inserted through an enzymatic cleavage site MfeI. A CMV promoter was used to initiate the expression of a fusion protein SV40 NLS-Cas-XX-NLS-GFP. A protein Cas-XX-NLS was linked to a green-fluorescent protein (GFP) through a linker peptide T2A. A promoter EF-1α was used to initiate the expression of a puromycin resistance gene. Plating: When growing to a confluency of 70% to 80%, CHO cells were collected and inoculated into a 12-well plate at 8*10\hat{ }4 cells/well. Transfection: 24 h after the plating, the transfection was conducted as follows: 6.25 μL of a Hieff Trans™ liposomal nucleic acid transfection reagent was added to 100 μL of opti-MEM, and thorough mixing was conducted. 2.5 g of a plasmid was added to 100 μL of opti-MEM, and thorough mixing was conducted. A diluted Hieff Trans™ liposomal nucleic acid transfection reagent was thoroughly mixed with a diluted plasmid, and incubation was conducted at room temperature for 20 min. A mixed solution produced after the incubation was added to a medium with cells for transfection. Addition of puromycin for screening: After the transfection was conducted for 24 h, the puromycin was added at a final concentration of 10 μg/mL. After the puromycin treatment was conducted for 24 h, culturing was further conducted for 24 h with a normal medium instead.

DNA was extracted, and a region near an editing region was amplified by PCR and sent for hiTOM sequencing: Cells were digested with trypsin, then collected, and subjected to genomic DNA extraction by a cell/tissue genomic DNA extraction kit (Bioteke). A region near a target on genomic DNA was amplified. A PCR product was subjected to hiTOM sequencing. Sequencing data analysis: Types and proportions of sequences within a range of 15 nt upstream of and 10 nt downstream of a target were counted, and sequences with a single-nucleotide variant (SNV) frequency of greater than or equal to 1% or a non-SNV mutation frequency of greater than or equal to 0.06% among the sequences were counted to obtain editing efficiencies of Cas-XX proteins for the target. A target sequence for the FUT8 gene in CHO cells was as follows:

FUT8-Cas-XX-g3:
(SEQ ID NO: 21)
TTCCAGCCAAGGTTGTGGACGGATCA,

where an italicized region was a PAM sequence and an underlined region was a targeting region. A gRNA sequence was

(SEQ ID NO: 22)
AGAGAAUGUGUGCAUAGUCAaCACCAGC
CAAGGUUGUGGACGGAUCA,

where an underlined region was a targeting region and the remaining region was a DR region.

Results were shown in FIG. 1. Compared to the WT Cas12i protein, the Cas12i protein with the N369R/S433R combined mutation demonstrated a significantly-improved editing efficiency, which was nearly two times an editing efficiency of the WT Cas12i protein. Editing types for the target gene included a base deletion, a base insertion, a base substitution, etc.

The acquisition and editing activity validation of the Cas12i mutant protein (S7R, D233R, D267R, N369R, and S433R) were conducted with reference to the Chinese patent application CN116004573A. Compared to SEQ ID NO: 1, in an amino acid sequence of the Cas12i (S7R, D233R, D267R, N369R, and S433R) mutant protein, S at position 7 was mutated into R, D at position 233 was mutated into R, D at position 267 was mutated into R, N at position 369 was mutated into R, and S at position 433 was mutated into R. The Cas12i (S7R, D233R, D267R, N369R, and S433R) mutant protein exhibited a significantly-higher editing efficiency than the WT Cas12i protein.

### Example 2 Production of Large-Seed and High-Yield Soybean by a Cas12i (N369R and S433R)-Based CRISPR Gene Editing Technology

### 1. Construction of a Gene-Editing Vector

The soybean genome includes 9 miR396 genes (miR396a/b/c/d/e/f/h/i/j). Sequence analysis shows that miR396a and miR396i have similar sequences, and miR396b, miR396c, miR396d, and miR396f have similar sequences. The members of the soybean miR396 (GmmiR396) family, miR396a, miR396b, miR396c, miR396d, miR396f, and miR396i, have nucleotide sequences shown in SEQ ID NOS: 2-7, respectively.

Sequences of soybean GmmiR396 were selected to design gRNAs. Guide sequences of the designed gRNAs were as follows:

| | Sequence | PAM | Targeted |
|---|---|---|---|
| gRNA | (5′-3′) | (5′-3′) | gene |
| gRNA1 | GGATGCAGTTCAAGA | TTT | GmmiR396a |
| | AAGCTGTGG | | (GmmiR396i |
| | (SEQ ID NO: 8) | | cannot be |
| | | | targeted) |
| gRNA2 | AGAAGTTCAAGAAAG | ATA | GmmiR396b/ |
| | CTGTGG | | c/d/f |
| | (SEQ ID NO: 9) | | |

DR for the above gRNAs was agagaaugugugcauagucacac (5′ to 3′, SEQ ID NO: 12). The gRNAs each included the DR and a respective guide sequence sequentially from 5′ to 3′.

Annealing primers were designed based on the target. After primer annealing, the gene-editing backbone vector was assembled through T5 exonuclease-mediated homologous recombination. The gene-editing vector carried a Cas12i (N369R and S433R) protein and the gRNA.

### 2. Acquisition of a Recombinant Strain

1) Transformation into Escherichia coli

The gene-editing vector obtained in the step 1 was transformed into Escherichia coli, and transformed Escherichia coli was subjected to bacterial-solution PCR. An amplification product with a correct PCR band size was selected for sequencing. Escherichia coli with a correct sequencing result was a recombinant Escherichia coli strain carrying the gene-editing vector.

2) Transformation into Agrobacterium

The recombinant Escherichia coli strain carrying the gene-editing vector obtained in the step 1) was cultured. Then the plasmid DNA was extracted, added to Agrobacterium competent cells, and placed in an ice bath for 5 min, in liquid nitrogen for 5 min, in a water bath at 37° C. for 5 min, and on ice for 5 min.

A centrifuge tube was taken out, 700 μL of a medium (without an antibiotic) was added, and shaking culture was conducted at 28° C. for 2 h to 4 h.

A resulting bacterial solution was collected and coated on a medium plate including a corresponding antibiotic, and invertedly cultured in an incubator. After about 2 d of the culturing, colonies could be observed. The colonies were subjected to PCR according to the method in the step 1). An amplification product was sequenced. Agrobacterium with a correct sequencing result was a recombinant Agrobacterium strain carrying the gene-editing vector.

### 3. Genetic Transformation of Plants

### 1) Sterilization of Soybean Seeds:

The selected soybean seeds (Zhonghuang 302) were placed in a petri dish in a dryer, 10 mL of concentrated hydrochloric acid was slowly added along a wall to a beaker with 150 mL of sodium hypochlorite in the dryer, and a lid of the dryer was quickly closed. The sterilization was conducted for 16 h with a chlorine gas generated from the sodium hypochlorite and concentrated hydrochloric acid.

### 2) Seed Germination:

The sterilized seeds obtained in the step 1) were inserted into a medium with a hilum facing downwards, and incubated for 24 h at 22° C. in the dark.

### 3) Preparation of an Agrobacterium Suspension:

A bacterial solution stored in a −80° C. freezer was taken, streaked on an antibiotic-containing YEP plate, and cultured at 28° C. for 2 d. Colonies were picked and inoculated into 5 mL of a YEP liquid medium in a 50 mL centrifuge tube, and cultured overnight at 28° C. under shaking. 300 μL of a resulting bacterial solution was taken and added to 250 mL of a YEP liquid medium, and cultured overnight until OD₆₀₀ was about 0.3 to 0.4. Centrifugation was conducted at 4,000 rpm for 10 min. Dilution was conducted with an infection solution to OD₆₀₀ of about 0.3 to 0.4 for later use.

### 4) Cotyledonary Node Scratching, Infection, and Co-Culture:

1 d after the seed germination in the step 2), a part of a hypocotyl was cut off, two cotyledons were separated, a half of a cotyledon that retained a cotyledonary node was clamped, two true leaves were removed, and 2 to 3 incisions were created at a growth point. The cut seeds were placed in an infection solution.

### 5) Recovery Culture:

An explant obtained after the co-culture was inserted into a recovery medium, and placed under light. The recovery culture was conducted at about 28° C. for 5 d to 7 d.

### 6) Screening Culture:

A part of a hypocotyl was cut off from a cotyledon after recovery, a resulting explant was inserted into a screening medium, and screening was then conducted at about 28° C. under light. Subculturing was conducted once every 10 d with 2 times in total.

### 7) Bud Elongation Culture:

Yellow leaves were removed from a selected explant, and a surface medium and rotted leaves were lightly tapped every 10 d to 15 d. A bottom surface of a cotyledonary node was cut off, and a treated cotyledonary node was inserted into an elongation medium (S5 medium) and cultured at 28° C. under light.

### 8) Rooting and Seedling Transplanting:

Seedlings growing to 3 cm to 4 cm during an elongation period were collected and placed on a rooting soil for rooting until at least three roots with a length of about 1 cm and three green leaves grew. Then, rooted plants were transplanted to an artificial light culture chamber for exercising. After about two weeks, when new leaves grew, resulting plants were transferred to a 28° C. artificial climate chamber and further cultured.

### 4. Detection and Phenotypic Observation for Transformed Soybean Plants

Edited seedlings were detected and selected from EO transformed seedlings through PCR and sequencing. With WT soybean (Zhonghuang 302) as a control, phenotypic changes were observed. A mutant population was expanded through 2 to 3 generations of self-crossing-based propagation to produce homozygous edited seedlings free of exogenous gene insertion.

### 5. Results

The above edited seedlings or edited plants were generated through gene editing with Zhonghuang 302 as a parent.

An editing type of the above gene-edited soybean was as follows:

Edited plant acd: The edited plant acd underwent mutations in miR396a, miR396c, and miR396d compared to the WT soybean (Zhonghuang 302), but had the consistent miR396b, miR396f, and miR396i with the WT soybean without mutations. In the edited plant acd, miR396a underwent deletions of 229th to 233rd bases (S bases in total) relative to a sequence shown in SEQ ID NO: 2, miR396c underwent deletions of 229th to 230th bases CT (2 bases in total) relative to a sequence shown in SEQ ID NO: 4, and miR396d underwent deletions of 19th to 29th bases (11 bases in total) relative to a sequence shown in SEQ ID NO: 5.

Edited plant acdf: The edited plant acdf underwent mutations in miR396a, miR396c, miR396d, and miR396f compared to the WT soybean (Zhonghuang 302), but had the consistent miR396b and miR396i with the WT soybean without mutations. In the edited plant acdf, miR396a underwent a deletion of a 229th base C relative to the sequence shown in SEQ ID NO: 2, miR396c underwent deletions of 230th to 255th bases (26 bases in total) relative to the sequence shown in SEQ ID NO: 4, miR396d underwent deletions of 19th to 29th bases (11 bases in total) relative to the sequence shown in SEQ ID NO: 5, and miR396f underwent a deletion of a 265th base T relative to a sequence shown in SEQ ID NO: 6.

Edited plant cdf: The edited plant cdf underwent mutations in miR396c, miR396d, and miR396f compared to the WT soybean (Zhonghuang 302), but had the consistent miR396a, miR396b, and miR396i with the WT soybean without mutations. In the edited plant cdf, miR396c underwent deletions of 228th to 253rd bases (26 bases in total) relative to the sequence shown in SEQ ID NO: 4, miR396d underwent a deletion of a 19th base C relative to the sequence shown in SEQ ID NO: 5, and miR396f underwent deletions of 264th to 265th bases (2 bases in total) relative to the sequence shown in SEQ ID NO: 6.

Edited plant abcdf: The edited plant abcdf underwent mutations in miR396a, miR396b, miR396c, miR396d, and miR396f compared to the WT soybean (Zhonghuang 302), but had the consistent miR396i with the WT soybean without mutations. In the edited plant abedf, miR396a underwent a deletion of a 229th base C relative to the sequence shown in SEQ ID NO: 2, miR396b underwent deletions of 30th to 100th bases (71 bases in total) relative to the sequence shown in SEQ ID NO: 3, miR396c underwent a deletion of a 229th base C relative to the sequence shown in SEQ ID NO: 4, miR396d underwent deletions of 18th to 26th bases (9 bases in total) relative to the sequence shown in SEQ ID NO: 5, and miR396f underwent deletions of 264th to 265th bases (2 bases in total) relative to the sequence shown in SEQ ID NO: 6.

Per-plant yields, numbers of seeds per plant, and hundred-seed weights of the WT soybean plant and the edited soybean plants cultivated in Hubei were measured, and average values of these results were as follows:

| | Per-plant | Number of seeds | Hundred-seed |
|---|---|---|---|
| Editing type | yield (g) | per plant | weight (g) |
| WT (Zhonghuang 302) | 37.0 | 190 | 19.5 |
| acd | 29.9 | 140.0 | 21.4 |
| acdf | 51.8 | 235.4 | 22.0 |
| cdf | 27.5 | 135.9 | 20.2 |
| abcdf | 44.8 | 203.1 | 22.1 |

It can be seen that, compared with the WT soybean plant (Zhonghuang 302), the edited soybean plants acd and cdf have a per-plant yield and a number of seeds per plant that do not increase, the edited soybean plants acd and cdf have a hundred-seed weight enhanced, and the edited soybean plants acdf and abodf have a per-plant yield, a number of seeds per plant, and a hundred-seed weight that all are significantly improved.

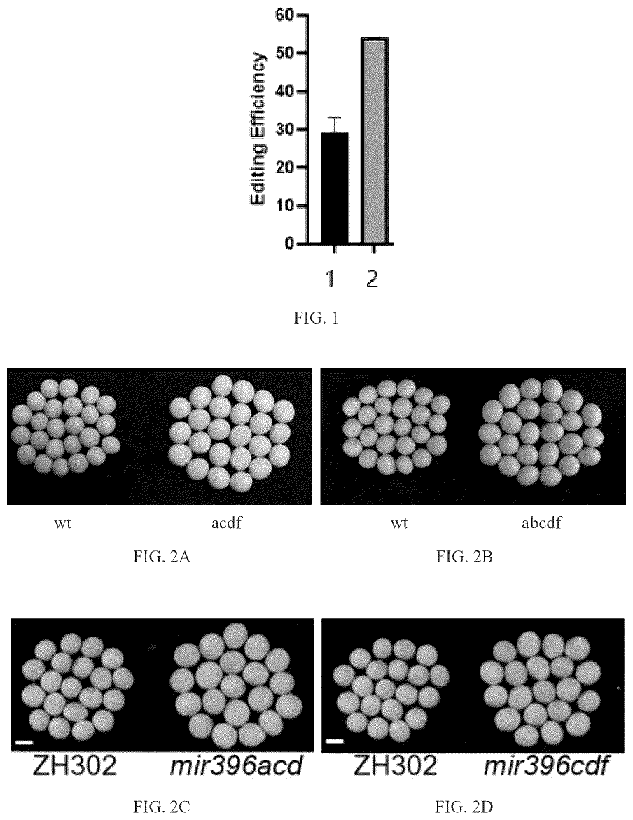

The comparison of seed sizes of the edited soybean plants acd, acdf, cdf, and abcdf with the WT soybean (Zhonghuang 302) is shown in FIGS. 2A-2D. A seed diameter of the edited soybean plant acdf is about 10.8% larger than a seed diameter of the WT soybean, a seed diameter of the edited soybean plant abcdf is about 15.3% larger than a seed diameter of the WT soybean, and seed sizes of the edited soybean plants acd and cdf are significantly larger than a seed size of the WT soybean.

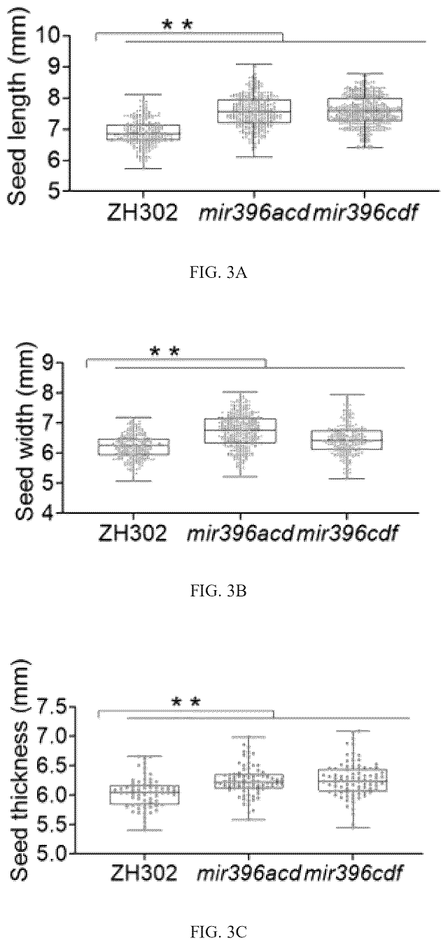

The comparison of seed lengths, seed widths, and seed thicknesses of the edited soybean plants acd and cdf with the WT soybean (Zhonghuang 302) is shown in FIGS. 3A-3C. Compared to the WT soybean, the edited soybean plants acd and cdf have a seed length, a seed width, and a seed thickness that all are significantly increased.

Therefore, compared with the WT soybean (Zhonghuang 302), the edited soybean plants acd, acdf, cdf, and abcdf all have a seed size increased.

### Example 3 Production of Large-Seed and High-Yield Soybean by a Cas12i (STR, D233R, D267R, N369R, and S433R)-Based CRISPR Gene Editing Technology

### 1. Construction of a Gene-Editing Vector

Sequences of soybean GmmiR396 were selected to design gRNAs. Guide sequences of the designed gRNAs were as follows:

| gRNA | Sequence(5′-3′) | PAM(5′-3′) | Targeted gene |
|---|---|---|---|
| gRNA3 | GTATTCTTCCACA | TTT | GmmiR396a/i |
| | GCTTTCTTGA | | |
| | (SEQ ID NO: 10) | | |
| ORNA4 | CTTTTCCACAGCTT | ATG | GmmiR396b/ |
| | TCTTGAACT | | c/d/f |
| | (SEQ ID NO: 11) | | |

DR for the above gRNAs was agagaaugugugcauagucacac (5′ to 3′, SEQ ID NO: 12). The gRNAs each included the DR and a respective guide sequence sequentially from S′ to 3′.

A Cas12i (S7R, D233R, D267R, N369R, and S433R) mutant protein and gRNAs 3-4 were adopted. Edited seedlings were prepared according to the method in Example 2. With WT soybean (Zhonghuang 302) as a control, phenotypic changes were observed. A mutant population was then expanded through 2 to 3 generations of self-crossing-based propagation to produce homozygous edited seedlings free of exogenous gene insertion.

The above edited seedlings or edited plants were generated through gene editing with Zhonghuang 302 as a parent.

An editing type of the above gene-edited soybean was as follows:

Edited plant aci: The edited plant aci underwent mutations in miR396a, miR396c, and miR396i compared to the WT soybean (Zhonghuang 302), but had the consistent miR396b, miR396d, and miR396f with the WT soybean without mutations. In the edited plant aci, miR396a underwent deletions of 229th to 233rd bases (5 bases in total) relative to a sequence shown in SEQ ID NO: 2, miR396c underwent deletions of 228th to 253th bases (26 bases in total) relative to a sequence shown in SEQ ID NO: 4, and miR396i underwent deletions of 200th to 201st bases (2 bases in total) relative to a sequence shown in SEQ ID NO: 7.

Edited plant adf: The edited plant adf underwent mutations in miR396a, miR396d, and miR396f compared to the WT soybean (Zhonghuang 302), but had the consistent miR396b, miR396c, and miR396i with the WT soybean without mutations. In the edited plant adf, miR396a underwent a deletion of a 229th base C relative to the sequence shown in SEQ ID NO: 2, miR396d underwent deletions of 19th to 29th bases CT (11 bases in total) relative to a sequence shown in SEQ ID NO: 5, and miR396f underwent a deletion of a 265th base T relative to a sequence shown in SEQ ID NO: 6.

Edited plant abcd: The edited plant abcd underwent mutations in miR396a, miR396b, miR396c, and miR396d compared to the WT soybean (Zhonghuang 302), but had the consistent miR396f and miR396i with the WT soybean without mutations. In the edited plant abcd, miR396a underwent deletions of 229th to 233rd bases (5 bases in total) relative to the sequence shown in SEQ ID NO: 2, miR396b underwent a deletion of a 29th base (1 base in total) relative to a sequence shown in SEQ ID NO: 3, miR396c underwent deletions of 229th to 230th bases (2 bases in total) relative to the sequence shown in SEQ ID NO: 4, and miR396d underwent deletions of 19th to 29th bases (11 bases in total) relative to the sequence shown in SEQ ID NO: 5.

Edited plant acfi: The edited plant acfi underwent mutations in miR396a, miR396c, miR396f, and miR396i compared to the WT soybean (Zhonghuang 302), but had the consistent miR396b and miR396d with the WT soybean without mutations. In the edited plant acfi, miR396a underwent deletions of 229th to 233rd bases (5 bases in total) relative to the sequence shown in SEQ ID NO: 2, miR396c underwent deletions of 228th to 253rd bases (26 bases in total) relative to the sequence shown in SEQ ID NO: 4, miR396f underwent a deletion of a 265th base T relative to the sequence shown in SEQ ID NO: 6, and miR396i underwent deletions of 200th to 201st bases (2 bases in total) relative to the sequence shown in SEQ ID NO: 7.

Edited plant bcdfi: The edited plant bedfi underwent mutations in miR396b, miR396c, miR396d, miR396f, and miR396i compared to the WT soybean (Zhonghuang 302), but had the consistent miR396a with the WT soybean without mutations. In the edited plant bedfi, miR396b underwent deletions of 29th to 31st bases (3 bases in total) relative to the sequence shown in SEQ ID NO: 3, miR396c underwent deletions of 229th to 230th bases (2 bases in total) relative to the sequence shown in SEQ ID NO: 4, miR396d underwent deletions of 18th to 26th bases (9 bases in total) relative to the sequence shown in SEQ ID NO: 5, miR396f underwent deletions of 264th to 265th bases (2 bases in total) relative to the sequence shown in SEQ ID NO: 6, and miR396i underwent deletions of 200th to 201st bases (2 bases in total) relative to the sequence shown in SEQ ID NO: 7.

Per-plant yields and hundred-seed weights of the WT soybean plant and the edited soybean plants cultivated in Hainan were measured, and average values of these results were as follows:

| | Per-plant | Hundred-seed |
|---|---|---|
| Editing type | yield (g) | weight (g) |
| WT (Zhonghuang 302) | 18.5 | 19.2 |
| aci | 20.8 | 20.5 |
| adf | 22.6 | 20.8 |
| abed | 24.0 | 21.5 |
| acfi | 20.8 | 20.3 |
| bcdfi | 25.2 | 22.1 |

It can be seen that, compared to the WT soybean plant (Zhonghuang 302), the edited soybean plants aci, adf, abcd, acfi, and bcdfi have a per-plant yield and a hundred-seed weight that both are improved.

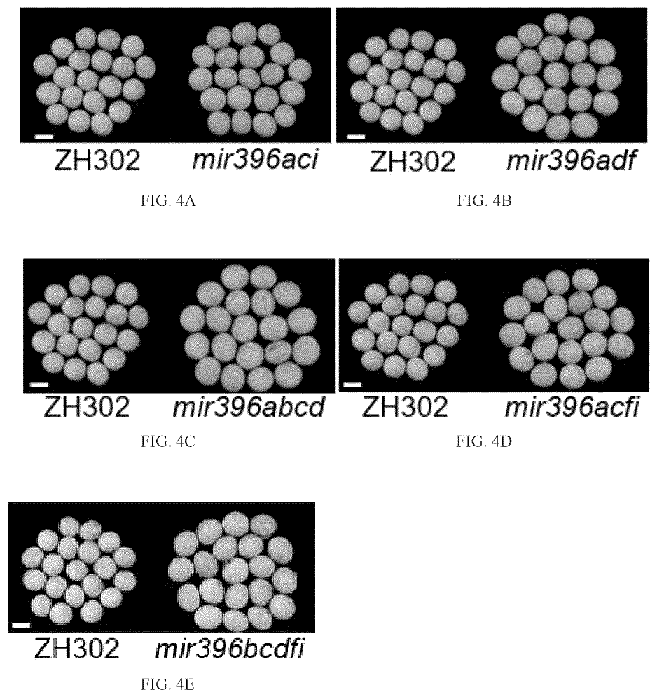

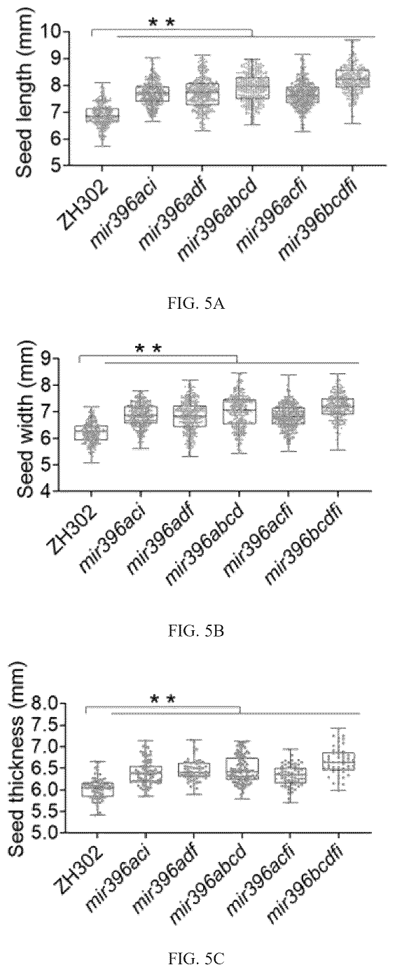

The comparison of seed sizes of the edited soybean plants aci, adf, abed, acfi, and bedfi with the WT soybean (Zhonghuang 302) is shown in FIGS. 4A-4E and FIGS. 5A-SC. Compared to the WT soybean, the edited soybean plants aci, adf, abcd, acfi, and bedfi have a seed size, a seed length, a seed width, and a seed thickness that all are significantly increased.

Compared to the WT soybean (Zhonghuang 302), the edited soybean plants aci, adf, abcd, acfi, and bcdfi have a seed size, a hundred-seed weight, and a yield that all are improved.

Editing types of the edited plants are summarized in the table below. The editing types in the following table refer to bases deleted in miR396a relative to the sequence shown in SEQ ID NO: 2, bases deleted in miR396b relative to the sequence shown in SEQ ID NO: 3, bases deleted in miR396c relative to the sequence shown in SEQ ID NO: 4, bases deleted in miR396d relative to the sequence shown in SEQ ID NO: 5, bases deleted in miR396f relative to the sequence shown in SEQ ID NO: 6, and bases deleted in miR396i relative to the sequence shown in SEQ ID NO: 7 in edited plants. WT refers to an edited plant in which miR396a, miR396b, miR396c, miR396d, miR396f, or miR396i is of WT and does not undergo any base mutation or deletion.

| Edited | Editing type | | | | | |
|---|---|---|---|---|---|---|
| plant | miR396a | miR396b | miR396c | miR396d | miR396f | miR396i |
| acd | 229th to 233rd | WT | 229th to 230th | 19th to 29th | WT | WT |
| aedf | 229th | WT | 230th to 255th | 19th to 29th | 265th | WT |
| cdf | WT | WT | 228th to 253rd | 19th | 264th to 265th | WT |
| abedf | 229th | 30th to 100th | 229th | 18th to 26th | 264th to 265th | WT |
| aci | 229th to 233rd | WT | 228th to 253rd | WT | WT | 200th to 201st |
| adf | 229th | WT | WT | 19th to 29th | 265th | WT |
| abed | 229th to 233rd | 29th | 229th to 230th | 19th to 29th | WT | WT |
| acfi | 229th to 233rd | WT | 228th to 253rd | WT | 265th | 200th to 201st |
| bcdfi | WT | 29th to 31st | 229th to 230th | 18th to 26th | 264th to 265th | 200th to 201st |

Trait manifestations of the edited plants (+ indicates that an edited plant undergoes a significant increase/improvement in a trait in contrast to the WT) are shown in the table below.

| Edited | Seed | Per-plant | Number | Hundred- |
|---|---|---|---|---|
| acd | + | | | + |
| aedf | + | + | + | + |
| cdf | + | | | + |
| abedf | + | + | + | + |
| aci | + | + | | + |
| adf | + | + | | + |
| abed | + | + | | + |
| acfi | + | + | | + |
| bcdfi | + | + | | + |

Additionally, the Cas12i mutant protein (N369R and S433R) and the Cas12i mutant protein (S7R, D233R, D267R, N369R, and S433R) are compared with the WT Cas12i protein for an editing efficiency targeting miR396. Results show that an editing efficiency of the WT Cas12i protein in soybean is only 1% to 2%, while editing efficiencies of the Cas12i mutant protein (N369R and S433R) and the Cas12i mutant protein (S7R, D233R, D267R, N369R, and S433R) in soybean exceed 10%.

Although the specific implementations of the present disclosure have been described in detail, those skilled in the art will understand that various modifications and changes can be made to the details according to all teachings published, and such modifications and changes all are within the protection scope of the present disclosure. The full content of the present disclosure is defined by the appended claims and any equivalents thereof.

What is claimed is:

1. A mutant microRNA396 (miR396), wherein the mutant miR396 undergoes a base mutation relative to parental miR396; the parental miR396 is wild-type (WT) miR396 derived from soybean; the miR396 is selected from one or more of miR396a, miR396b, miR396c, miR396d, miR396f, and miR396i; and
the mutant miR396 is configured to improve a seed size, a seed weight, a number of seeds per plant, or a yield for the soybean.

2. The mutant miR396 according to claim 1, wherein the mutant miR396 undergoes a base deletion relative to the parental miR396; the parental miR396 is the WT miR396 derived from the soybean; the miR396 is selected from one of the following (1) to (9):
(1) the miR396 comprises the miR396a, the miR396c, and the miR396d;
(2) the miR396 comprises the miR396a, the miR396c, the miR396d, and the miR396f;
(3) the miR396 comprises the miR396c, the miR396d, and the miR396f;
(4) the miR396 comprises the miR396a, the miR396b, the miR396c, the miR396d, and the miR396f;
(5) the miR396 comprises the miR396a, the miR396c, and the miR396i;
(6) the miR396 comprises the miR396a, the miR396d, and the miR396f;
(7) the miR396 comprises the miR396a, the miR396b, the miR396c, and the miR396d;
(8) the miR396 comprises the miR396a, the miR396c, the miR396f, and the miR396i; and
(9) the miR396 comprises the miR396b, the miR396c, the miR396d, the miR396f, and the miR396i; and
preferably, the mutant miR396 is selected from one or more of the following (i) to (ix):
(i) mutant miR396a undergoing a base deletion relative to the sequence shown in SEQ ID NO: 2; mutant miR396b undergoing a base deletion relative to the sequence shown in SEQ ID NO: 3; mutant miR396c undergoing a base deletion relative to the sequence shown in SEQ ID NO: 4; mutant miR396d undergoing a base deletion relative to the sequence shown in SEQ ID NO: 5; and mutant miR396f undergoing a base deletion relative to the sequence shown in SEQ ID NO: 6;
(ii) the mutant miR396b undergoing the base deletion relative to the sequence shown in SEQ ID NO: 3; the mutant miR396c undergoing the base deletion relative to the sequence shown in SEQ ID NO: 4; the mutant miR396d undergoing the base deletion relative to the sequence shown in SEQ ID NO: 5; the mutant miR396f undergoing the base deletion relative to the sequence shown in SEQ ID NO: 6; and mutant miR396i undergoing a base deletion relative to a sequence shown in SEQ ID NO: 7;
(iii) the mutant miR396a undergoing the base deletion relative to the sequence shown in SEQ ID NO: 2; the mutant miR396c undergoing the base deletion relative to the sequence shown in SEQ ID NO: 4; and the mutant miR396d undergoing the base deletion relative to the sequence shown in SEQ ID NO: 5;
(iv) the mutant miR396a undergoing the base deletion relative to the sequence shown in SEQ ID NO: 2; the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(v) the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(vi) the mutant miR396a undergoing the base deletion relative to the sequence shown in SEQ ID NO: 2; the mutant miR396c undergoing the base deletion relative to the sequence shown in SEQ ID NO: 4; and the mutant miR396i undergoing the base deletion relative to the sequence shown in SEQ ID NO: 7;
(vii) the mutant miR396a undergoing the base deletion relative to the sequence shown in SEQ ID NO: 2; the mutant miR396d undergoing the base deletion relative to the sequence shown in SEQ ID NO: 5; and the mutant miR396f undergoing the base deletion relative to the sequence shown in SEQ ID NO: 6;
(viii) the mutant miR396a undergoing the base deletion relative to the sequence shown in SEQ ID NO: 2; the mutant miR396b undergoing the base deletion relative to the sequence shown in SEQ ID NO: 3; the mutant miR396c undergoing the base deletion relative to the sequence shown in SEQ ID NO. 4; and the mutant miR396d undergoing the base deletion relative to the sequence shown in SEQ ID NO: 5; and
(ix) the mutant miR396a undergoing the base deletion relative to the sequence shown in SEQ ID NO: 2; the mutant miR396c undergoing the base deletion relative to the sequence shown in SEQ ID NO: 4; the mutant miR396f undergoing the base deletion relative to the sequence shown in SEQ ID NO: 6; and the mutant miR396i undergoing the base deletion relative to the sequence shown in SEQ ID NO: 7.

3. A gene editing reagent, wherein the gene editing reagent is configured to cause generation of the mutant miR396 according to claim 1 in the soybean; the gene editing reagent comprises Cas12i and guide RNA (gRNA); the gRNA comprises a scaffold region for binding to the Cas12i and a guide sequence for hybridizing with a target sequence; the gRNA targets miR396 in the soybean; and
preferably, an amino acid sequence of the Cas12i undergoes mutations of 369th and 433rd amino acids relative to the sequence shown in SEQ ID NO: 1; or
the amino acid sequence of the Cas12i undergoes mutations of 7th, 233rd, 267th, 369th, and 433rd amino acids relative to the sequence shown in SEQ ID NO: 1.

4. (canceled)

5. A method for producing soybean with an improved seed size, seed weight, number of seeds per plant, or yield, comprising the following steps:
(a) introducing the mutant miR396 according to claim 1 into a soybean cell, a soybean seed, a soybean tissue, or a soybean part; and
(b) regenerating a soybean plant from a soybean cell, a soybean seed, a soybean tissue, or a soybean part produced in the step (a).

6. The method according to claim 5, wherein the introducing the mutant miR396 comprises a step of mutating endogenous miR396 of the soybean to introduce the mutant miR396; and
preferably, the mutant miR396 is introduced into the soybean cell, the soybean seed, the soybean tissue, or the soybean part through gene editing.

7. The method according to claim 6, wherein the gene editing is conducted with Cas12i in the soybean cell, the soybean seed, the soybean tissue, or the soybean part; and
preferably, an amino acid sequence of the Cas12i undergoes mutations of 369th and 433rd amino acids relative to the sequence shown in SEQ ID NO: 1; or
the amino acid sequence of the Cas12i undergoes mutations of 7th, 233rd, 267th, 369th, and 433rd amino acids relative to the sequence shown in SEQ ID NO: 1.

8. A method for gene editing of soybean, comprising the following steps:
(a) conducting the gene editing with Cas12i and gRNA in a soybean cell, a soybean seed, a soybean tissue, or a soybean part to produce a gene-edited soybean cell, soybean seed, soybean tissue, or soybean part, wherein the gRNA comprises a scaffold region for binding to the Cas12i and a guide sequence for hybridizing with a target sequence, and the gRNA targets miR396 in the soybean; and
(b) regenerating a soybean plant from the gene-edited soybean cell, soybean seed, soybean tissue, or soybean part obtained in the step (a),
wherein preferably, an amino acid sequence of the Cas12i undergoes mutations of 369th and 433rd amino acids relative to the sequence shown in SEQ ID NO: 1; or
the amino acid sequence of the Cas12i undergoes mutations of 7th, 233rd, 267th, 369th, and 433rd amino acids relative to the sequence shown in SEQ ID NO: 1.

9. The method according to claim 8, wherein the guide sequence for hybridizing with the target sequence in the gRNA is shown in one of SEQ ID NOS: 8-11.

10. A method for producing soybean with an improved seed size, seed weight, number of seeds per plant, or yield, comprising a step of hybridizing a soybean seed or the soybean plant prepared by the method according to claim 5 with another soybean variety to produce the soybean with the improved seed size, seed weight, number of seeds per plant, or yield.

11. The gene editing reagent according to claim 3, wherein the mutant miR396 undergoes a base deletion relative to the parental miR396; the parental miR396 is the WT miR396 derived from the soybean; the parental miR396 is selected from one of the following (1) to (9):
(1) the parental miR396 comprises the miR396a, the miR396c, and the miR396d;
(2) the parental miR396 comprises the miR396a, the miR396c, the miR396d, and the miR396f;
(3) the parental miR396 comprises the miR396c, the miR396d, and the miR396f;
(4) the parental miR396 comprises the miR396a, the miR396b, the miR396c, the miR396d, and the miR396f;
(5) the parental miR396 comprises the miR396a, the miR396c, and the miR396i,
(6) the parental miR396 comprises the miR396a, the miR396d, and the miR396f;
(7) the parental miR396 comprises the miR396a, the miR396b, the miR396c, and the miR396d;
(8) the parental miR396 comprises the miR396a, the miR396c, the miR396f, and the miR396i; and
(9) the parental miR396 comprises the miR396b, the miR396c, the miR396d, the miR396f, and the miR396i; and
the mutant miR396 is selected from one or more of the following (i) to (ix):
(i) mutant miR396a undergoing a base deletion relative to the sequence shown in SEQ ID NO: 2; mutant miR396b undergoing a base deletion relative to the sequence shown in SEQ ID NO: 3; mutant miR396c undergoing a base deletion relative to the sequence shown in SEQ ID NO: 4; mutant miR396d undergoing a base deletion relative to the sequence shown in SEQ ID NO: 5; and mutant miR396f undergoing a base deletion relative to the sequence shown in SEQ ID NO: 6;
(ii) the mutant miR396b undergoing the base deletion relative to the sequence shown in SEQ ID NO: 3; the mutant miR396c undergoing the base deletion relative to the sequence shown in SEQ ID NO: 4; the mutant miR396d undergoing the base deletion relative to the sequence shown in SEQ ID NO: 5; the mutant miR396f undergoing the base deletion relative to the sequence shown in SEQ ID NO: 6; and mutant miR396i undergoing a base deletion relative to a sequence shown in SEQ ID NO: 7;
(iii) the mutant miR396a undergoing the base deletion relative to the sequence shown in SEQ ID NO: 2; the mutant miR396c undergoing the base deletion relative to the sequence shown in SEQ ID NO: 4; and the mutant miR396d undergoing the base deletion relative to the sequence shown in SEQ ID NO: 5;
(iv) the mutant miR396a undergoing the base deletion relative to the sequence shown in SEQ ID NO: 2; the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(v) the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(vi) the mutant miR396a undergoing the base deletion relative to the sequence shown in SEQ ID NO: 2; the mutant miR396c undergoing the base deletion relative to the sequence shown in SEQ ID NO: 4; and the mutant miR396i undergoing the base deletion relative to the sequence shown in SEQ ID NO: 7;
(vii) the mutant miR396a undergoing the base deletion relative to the sequence shown in SEQ ID NO: 2; the mutant miR396d undergoing the base deletion relative to the sequence shown in SEQ ID NO: 5; and the mutant miR396f undergoing the base deletion relative to the sequence shown in SEQ ID NO: 6;
(viii) the mutant miR396a undergoing the base deletion relative to the sequence shown in SEQ ID NO: 2, the mutant miR396b undergoing the base deletion relative to the sequence shown in SEQ ID NO: 3; the mutant miR396c undergoing the base deletion relative to the sequence shown in SEQ ID NO: 4; and the mutant miR396d undergoing the base deletion relative to the sequence shown in SEQ ID NO: 5; and
(ix) the mutant miR396a undergoing the base deletion relative to the sequence shown in SEQ ID NO: 2; the mutant miR396c undergoing the base deletion relative to the sequence shown in SEQ ID NO: 4; the mutant miR396f undergoing the base deletion relative to the sequence shown in SEQ ID NO: 6; and the mutant miR396i undergoing the base deletion relative to the sequence shown in SEQ ID NO: 7.

12. The method according to claim 5, wherein,
a seed size of soybean carrying the mutant miR396 is at least 3% larger than a seed size of parental soybean; or,
a single-seed weight, hundred-seed weight, or thousand-seed weight of the soybean carrying the mutant miR396 is at least 5% higher than a single-seed weight, hundred-seed weight, or thousand-seed weight of the parental soybean; or,
a number of seeds per plant of the soybean carrying the mutant miR396 is at least 3% larger than a number of seeds per plant of the parental soybean; or,
a yield, per-plant yield, or per-mu yield of the soybean carrying the mutant miR396 is at least 5% higher than a yield, per-plant yield, or per-mu yield of the parental soybean.

13. The method according to claim 5, wherein the mutant miR396 undergoes a base deletion relative to the parental miR396; the parental miR396 is the WT miR396 derived from the soybean; the parental miR396 is selected from one of the following (1) to (9):
(1) the parental miR396 comprises the miR396a, the miR396c, and the miR396d;
(2) the parental miR396 comprises the miR396a, the miR396c, the miR396d, and the miR396f;
(3) the parental miR396 comprises the miR396c, the miR396d, and the miR396f;
(4) the parental miR396 comprises the miR396a, the miR396b, the miR396c, the miR396d, and the miR396f;
(5) the parental miR396 comprises the miR396a, the miR396c, and the miR396i;
(6) the parental miR396 comprises the miR396a, the miR396d, and the miR396f;
(7) the parental miR396 comprises the miR396a, the miR396b, the miR396c, and the miR396d;
(8) the parental miR396 comprises the miR396a, the miR396c, the miR396f, and the miR396i; and
(9) the parental miR396 comprises the miR396b, the miR396c, the miR396d, the miR396f, and the miR396i; and
the mutant miR396 is selected from one or more of the following (i) to (ix):
(i) mutant miR396a undergoing a base deletion relative to the sequence shown in SEQ ID NO: 2; mutant miR396b undergoing a base deletion relative to the sequence shown in SEQ ID NO: 3; mutant miR396c undergoing a base deletion relative to the sequence shown in SEQ ID NO: 4; mutant miR396d undergoing a base deletion relative to the sequence shown in SEQ ID NO: 5; and mutant miR396f undergoing a base deletion relative to the sequence shown in SEQ ID NO: 6;
(ii) the mutant miR396b undergoing the base deletion relative to the sequence shown in SEQ ID NO: 3; the mutant miR396c undergoing the base deletion relative to the sequence shown in SEQ ID NO: 4; the mutant miR396d undergoing the base deletion relative to the sequence shown in SEQ ID NO: 5; the mutant miR396f undergoing the base deletion relative to the sequence shown in SEQ ID NO: 6; and mutant miR396i undergoing a base deletion relative to a sequence shown in SEQ ID NO: 7;
(iii) the mutant miR396a undergoing the base deletion relative to the sequence shown in SEQ ID NO: 2; the mutant miR396c undergoing the base deletion relative to the sequence shown in SEQ ID NO: 4; and the mutant miR396d undergoing the base deletion relative to the sequence shown in SEQ ID NO: 5;
(iv) the mutant miR396a undergoing the base deletion relative to the sequence shown in SEQ ID NO: 2; the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(v) the mutant miR396c undergoing the base deletion relative to the sequence shown in SEQ ID NO: 4; the mutant miR396d undergoing the base deletion relative to the sequence shown in SEQ ID NO: 5; and the mutant miR396f undergoing the base deletion relative to the sequence shown in SEQ ID NO: 6;
(vi) the mutant miR396a undergoing the base deletion relative to the sequence shown in SEQ ID NO: 2; the mutant miR396c undergoing the base deletion relative to the sequence shown in SEQ ID NO: 4, and the mutant miR396i undergoing the base deletion relative to the sequence shown in SEQ ID NO: 7;
(vii) the mutant miR396a undergoing the base deletion relative to the sequence shown in SEQ ID NO: 2; the mutant miR396d undergoing the base deletion relative to the sequence shown in SEQ ID NO: 5; and the mutant miR396f undergoing the base deletion relative to the sequence shown in SEQ ID NO: 6;
(viii) the mutant miR396a undergoing the base deletion relative to the sequence shown in SEQ ID NO: 2; the mutant miR396b undergoing the base deletion relative to the sequence shown in SEQ ID NO: 3; the mutant miR396c undergoing the base deletion relative to the sequence shown in SEQ ID NO: 4; and the mutant miR396d undergoing the base deletion relative to the sequence shown in SEQ ID NO: 5; and
(ix) the mutant miR396a undergoing the base deletion relative to the sequence shown in SEQ ID NO: 2; the mutant miR396c undergoing the base deletion relative to the sequence shown in SEQ ID NO: 4; the mutant miR396f undergoing the base deletion relative to the sequence shown in SEQ ID NO: 6; and the mutant miR396i undergoing the base deletion relative to the sequence shown in SEQ ID NO: 7.

14. The method according to claim 13, wherein the introducing the mutant miR396 comprises a step of mutating endogenous miR396 of the soybean to introduce the mutant miR396; and
the mutant miR396 is introduced into the soybean cell, the soybean seed, the soybean tissue, or the soybean part through gene editing.

15. The method according to claim 14, wherein the gene editing is conducted with Cas12i in the soybean cell, the soybean seed, the soybean tissue, or the soybean part; and
an amino acid sequence of the Cas12i undergoes mutations of 369th and 433rd amino acids relative to the sequence shown in SEQ ID NO: 1; or
the amino acid sequence of the Cas12i undergoes mutations of 7th, 233rd, 267th, 369th, and 433rd amino acids relative to the sequence shown in SEQ ID NO: 1.

16. The method according to claim 10, wherein the introducing the mutant miR396 comprises a step of mutating endogenous miR396 of the soybean to introduce the mutant miR396; and
the mutant miR396 is introduced into the soybean cell, the soybean seed, the soybean tissue, or the soybean part through gene editing.

17. The method according to claim 16, wherein the gene editing is conducted with Cas12i in the soybean cell, the soybean seed, the soybean tissue, or the soybean part; and
an amino acid sequence of the Cas12i undergoes mutations of 369th and 433rd amino acids relative to the sequence shown in SEQ ID NO: 1; or
the amino acid sequence of the Cas12i undergoes mutations of 7th, 233rd, 267th, 369th, and 433rd amino acids relative to the sequence shown in SEQ ID NO: 1.

18. A method for producing soybean with an improved seed size, seed weight, number of seeds per plant, or yield, comprising a step of hybridizing a soybean seed or the soybean plant prepared by the method according to claim 8 with another soybean variety to produce the soybean with the improved seed size, seed weight, number of seeds per plant, or yield.

19. The method according to claim 18, wherein the guide sequence for hybridizing with the target sequence in the gRNA is shown in one of SEQ ID NOS: 8-11.


SEQUENCE LISTING (22 sequences)

SEQ ID NO: 1 - protein, 1045 aa, unidentified
MKKVEVSRPYQSLLLPNHRKFKYLDETWNAYKSVKSLLHRFLVCAYGAVPFNKFVEVVEKVDNDQLVLAFAVRLFRLVPVESTSFAKVDKANLAKSLANHLPVGTAIPANVQSYFDSNFDPKKYMWIDCAWEADRLAREMGLSASQFSEYATTMLWEDWLPLNKDDVNGWGSVSGLFGEGKKEDRQQKVKMLNNLLNGIKKNPPKDYTQYLKILLNAFDAKSHKEAVKNYKGDSTGRTASYLSEKSGEITELMLEQLMSNIQRDIGDKQKEISLPKKDVVKKYLESESGVPYDQNLWSQAYRNAASSIKKTDTRNFNSTLEKFKNEVELRGLLSEGDDVEILRSKFFSSEFHKTPDKFVIKPEHIGFNNKYNVVAELYKLKAEATDFESAFATVKDEFEEKGIKHPIKNILEYIWNNEVPVEKWGRVARFNQSEEKLLRIKANPTVECNQGMTFGNSAMVGEVLRSNYVSKKGALVSGEHGGRLIGQNNMIWLEMRLLNKGKWETHHVPTHNMKFFEEVHAYNPSLADSVNVRNRLYRSEDYTQLPSSITDGLKGNPKAKLLKRQHCALNNMTANVLNPKLSFTINKKNDDYTVIIVHSVEVSKPRREVLVGDYLVGMDQNQTASNTYAVMQVVKPKSTDAIPFRNMWVRFVESGSIESRTLNSRGEYVDQLNHDGVDLFEIGDTEWVDSARKFFNKLGVKHKDGTLVDLSTAPRKAYAFNNFYFKTMLNHLRSNEVDLTLLRNEILRVANGRFSPMRLGSLSWTTLKALGSFKSLVLSYFDRLGAKEMVDKEAKDKSLFDLLVAINNKRSNKREERTSRIASSLMTVAQKYKVDNAVVHVVVEGNLSSTDRSASKAHNRNTMDWCSRAVVKKLEDMCNLYGFNIKGVPAFYTSHQDPLVHRADYDDPKPALRCRYSSYSRADFSKWGQNALAAVVRWASNKKSNTCYKVGAVEFLKQHGLFADKKLTVEQFLSKVKDEEILIPRRGGRVFLTTHRLLAESTFVYLNGVKYHSCNADEVAAVNICLNDWVIPCKKKMKEESSASG

SEQ ID NO: 2 - RNA, 540 nt, unidentified
  source 1..540: mol_type = other RNA
aaataccataccatctcctcccctacacctacaccttttctctctcttagcttagccttctcctattctggacctctgtcccttcctcttgattagggttctaattatgttatgaaccttccatgcttttatttttcttttaaaaaaagaataaagaaaaccctacatatgggtttgtggggtatattcagtcttcaatatcatggctctctttgtattcttccacagctttcttgaactgcatccaaagagttcctttgcatgcatgccatggcactcttactcccaaatcttgttttgcggttcaataaagctgtgggaagatacagatagggtcaacaacggactagcttatctcaaactccagattcttctctaaatcttcctatccaattatatatagggataaaattattgcaaagtattttttttcctttttcttttgtgatttgacttaatttaaatcattttcagatttaaaccatcgacttttcagatatatctctatatgtgatcagaattctcagatgcagtattatg

SEQ ID NO: 3 - RNA, 126 nt, unidentified
  source 1..126: mol_type = other RNA
ctcaagtcctggtcatgcttttccacagctttcttgaacttcttatgcatcttatatctctccacctccaggattttaagccctagaagctcaagaaagctgtgggagaatatggcaattcaggct

SEQ ID NO: 4 - RNA, 517 nt, unidentified
  source 1..517: mol_type = other RNA
ctggctgcacctttcatattgcaatgcagccccactgtccctgatcctgatcttatctttatatcatgttgaaatgttgatattgcttatctacagcaagctcaaagggtcttcctcttctctcctttccactcctctataattcaattaattctgctatatatttggctgcttcaggatttcaacttccatgctttcttcaacaagtcctgttatgcttttccacagctttcttgaacttcttatgcctagtgcaattattgatgtggcatagaagtttaagaaaaatgtggaaaaacatgtcaaatctaggacttaattggttctaaaaaatcccacacaataagcttcttcttttttcagttattgcatatgccacaaataaatggtggtctaattggaaggagatcatcaaactcatcccacaataatatgaattcaaatttcactaccaatatgaatactatgtagatgaacgatttgtaagtgttttataagtattctttaactcacctaa

SEQ ID NO: 5 - RNA, 108 nt, unidentified
  source 1..108: mol_type = other RNA
ggtcatgcttttccacagctttcttgaacttcttatgcatcttatatctctccacttccagcattttaagccctagaagctcaagaaagctgtgggagaatatggcaa

SEQ ID NO: 6 - RNA, 551 nt, unidentified
  source 1..551: mol_type = other RNA
gctgatgttgcagtagtaaacttgtgaattaactgtggctggctgcacctttcaaattgcaatgcagccccactgtccctgatcctgatcttatcttttatatgatgttgaaatgttgattgcttatctacagcaagctcaaagggtcttcctcctctctcctttccactcctctaactcaattcattctgctatatatttagcttcttcagcatttcaacttccatgcttgcttgaacaagtcctgttatgcttttccacagctttcttgaacttcttatgcctaatgcagctattgatgtggcattgaagtttaagaaaaatgtggaaaaacatgtcaaatctaggacttaattggttataagaacttacacagtacacctttttttttctcacagcatatgtcgcggacaaaggtctcactgtgtcaatatgaagactttgttgataaatcatttgtaaatatctttataagtattctttgaatcttgagatatatactgatccatccatgtcaaaaaaaaaatttcatcacacagaaaagccatagc

SEQ ID NO: 7 - RNA, 1309 nt, unidentified
  source 1..1309: mol_type = other RNA
tctctctcttagcttggcgttctcttattctggacctctctcccttccctcttgattagggttctgattatgttatgaaccttcaatgattcttttttttcttcaaagaaaacaaagagaaagaaaaccctatatatgggcttgtggagtatttatcttcagtcttcaatatcatggccctctttgtattcttccacagctttcttgaactgcatccatagagttcctttgcatgcatgccaaggcactcttgctcccacaccttgttttgcggttcaataaagctgtgggaagatacagatagggtcaacaacaacgaactagcttatctcaaactccagattcctctctaaagcttcctatccaattgtatatatatatatatatatatatatatatagggataaattattgcaatgtatcttttttcctttatcttttgtgatttgacttaaatctttttcagatttaagacattgaattttcagatatatatctctattgtgtgatcagaattctcagatgcagtattttgtgagagaataaacttgtaccagttgggtagatttgtgactatgaggatgatgattccccaacaaattggcttcttcttctggaattaaacggatcaggtatcgattcgatctatgtaaaatcaagggaatttcggctcacgattttggtagatgcggaatcactatgcaaactttattttcagatcttataataattaatatgtttgaaatccgttatatatatgtgtgtgtgtttgccttaaattttatatcccaattgaatttcctcttaattaactttggccagcaattccaggcttaaacgagaggggaacatatggtagtggttaatttgcacatgaaaactattgatttatgataatgcatcgtataggcgtagctgtttgatttgaaaatattttttcttttttgtgttctagctagctagtcctgtctgtgcagaatataataattataattttaataatttcagcgttagctgcttctgctctttttctcgtctctctgctcactcaccatattagcctttttcttttgttttgaaatggtcttttgtgtagcctacaaatgcaggctaaagtttcaacttacagtggatcaatggtcatcattgagatttctccatagaacatgtctcactcaattcattaaacctttgatttgtgtacttgtactgttttagtcggcaaagtcacagatagaattcagtaatttaatatatattccagcctgcagattcagcataaccacaagttagtatctattaattaatc

SEQ ID NO: 8 - RNA, 24 nt, synthetic construct
  source 1..24: mol_type = other RNA
  misc_feature 1..24: note = gRNA
ggatgcagttcaagaaagctgtgg

SEQ ID NO: 9 - RNA, 21 nt, synthetic construct
  source 1..21: mol_type = other RNA
  misc_feature 1..21: note = gRNA
agaagttcaagaaagctgtgg

SEQ ID NO: 10 - RNA, 23 nt, synthetic construct
  source 1..23: mol_type = other RNA
  misc_feature 1..23: note = gRNA
gtattcttccacagctttcttga

SEQ ID NO: 11 - RNA, 23 nt, synthetic construct
  source 1..23: mol_type = other RNA
  misc_feature 1..23: note = gRNA
cttttccacagctttcttgaact

SEQ ID NO: 12 - RNA, 23 nt, synthetic construct
  source 1..23: mol_type = other RNA
  misc_feature 1..23: note = gRNA-DR
agagaatgtgtgcatagtcacac

SEQ ID NO: 13 - RNA, 24 nt, synthetic construct
  source 1..24: mol_type = other RNA
agagaatgtgtgcatagtcaacac

SEQ ID NO: 14 - RNA, 24 nt, synthetic construct
  source 1..24: mol_type = other RNA
agagaatgtgtgcatagtctacac

SEQ ID NO: 15 - RNA, 24 nt, synthetic construct
  source 1..24: mol_type = other RNA
agagaatgtgtgcatagtccacac

SEQ ID NO: 16 - RNA, 24 nt, synthetic construct
  source 1..24: mol_type = other RNA
agagaatgtgtgcatagtcgacac

SEQ ID NO: 17 - DNA, 42 bp, synthetic construct
  source 1..42: mol_type = other DNA
cggctttaacaggaagtacaacgtggtcgccgagctgtacaa

SEQ ID NO: 18 - DNA, 40 bp, synthetic construct
  source 1..40: mol_type = other DNA
ttgtacttcctgttaaagccgatgtgctctggcttgatca

SEQ ID NO: 19 - DNA, 39 bp, synthetic construct
  source 1..39: mol_type = other DNA
cggtttaaccagcgggaggagaagctgctgcggatcaag

SEQ ID NO: 20 - DNA, 39 bp, synthetic construct
  source 1..39: mol_type = other DNA
tcctcccgctggttaaaccgggccaccctgccccacttc

SEQ ID NO: 21 - DNA, 26 bp, synthetic construct
  source 1..26: mol_type = other DNA
ttccagccaaggttgtggacggatca

SEQ ID NO: 22 - RNA, 47 nt, synthetic construct
  source 1..47: mol_type = other RNA
agagaatgtgtgcatagtcaacaccagccaaggttgtggacggatca